(19) United States
(12) Patent Application Publication
(10) Pub. No.: US 2020/0085021 A1
(43) Pub. Date: Mar. 19, 2020

(54) MODEL SYSTEM FOR MITOCHONDRIAL DYSFUNCTION AND METHODS OF USING THE SAME

(71) Applicant: The UAB Research Foundation, Birmingham, AL (US)

(72) Inventor: Keshav K SINGH, Birmingham, AL (US)

(21) Appl. No.: 16/573,705

(22) Filed: Sep. 17, 2019

Related U.S. Application Data
(60) Provisional application No. 62/732,249, filed on Sep. 17, 2018.

(51) Int. Cl. A01K 67/027; C12N 15/85

(52) U.S. Cl. CPC A01K 67/0278; C12N 15/85; A01K 2207/15; A01K 2217/203; A01K 2217/206; A01K 2267/0393; C12N 2015/859; A01K 2267/0306; A01K 2227/105

(57) ABSTRACT

A transgenic non-human animal model is disclosed. The system comprises an inducible transgene allowing the expression of a mutant POLG1 polypeptide that results in modulation of mitochondrial DNA copy number and/or concentration in the whole transgenic non-human animal or selected cells or tissues of the transgenic non-human animal. Methods of producing and using the model system are also provided.

### GOVERNMENT SUPPORT CLAUSE

“This invention was made with government support under RO 1 CA204430 awarded by National Institutes of Health and 1101BX001716 awarded by the Veterans Administration. The government has certain rights in the invention.” 37 CFR 401.14(f)(4).

### FIELD OF THE DISCLOSURE

The present disclosure relates to a transgenic non-human animal model of mitochondrial dysfunction. More specifically, the present disclosure relates to a transgenic non-human animal model of mitochondrial dysfunction in which mitochondrial DNA is depleted and/or repleted in a controlled manner as well as methods of producing and using the transgenic non-human animal model.

### BACKGROUND OF THE DISCLOSURE

Mitochondrial DNA (mtDNA) depletion is involved in many diseases and conditions, such as mtDNA depletion syndromes, mitochondrial diseases, skin wrinkles and other changes in the skin, hair loss, aging and aging-associated chronic diseases, and other human pathologies. Fundamental questions about mitochondrial biology and mtDNA biology remain mostly unsolved. To answer these questions, appropriate animal models are required. Therefore, animal models capable of inducing mitochondrial dysfunction and/or modulating mtDNA copy number and/or concentration are crucial tools for understanding mitochondrial pathology. However, engineering mtDNA has proved challenging owing to the multicopy nature of the mitochondrial genome. Furthermore, the transfection of plasmids or modified mtDNA into animal mitochondria has met with varying success.

Transplantation of stem cells is an attractive strategy for cell replacement in trauma and a wide range of disorders which are associated with cell/tissue degeneration. This cell replacement approach is particularly relevant for disorders of the brain and spinal cord. A major issue in stem cell transplantation is to control the differentiation of the stem cells to the desired cell type (both in vitro and in vivo) type of cells. Generally, stem cell differentiation in vitro is more predictable than stem cell differentiation in vivo.

The art is in need of new animal models to evaluate the consequences of mitochondrial dysfunction, such as, but not limited to, mtDNA depletion, particularly as it occurs during the aging. Such animal models are currently lacking in the art. The present disclosure provides a novel transgenic non-human animal model in which mtDNA content can be modulated in a controlled manner to provide for mtDNA depletion as well as mtDNA repletion. The disclosed animal model may be used to evaluate the consequences of depleted and/or repleted mtDNA copy number and/or concentration in the whole animal, in selected cells/tissues, or in a developmentally specific manner. Further, the transgenic non-human animal model may be used to define the pathway(s) involved in mtDNA function, and to identify and develop therapeutics for the treatment of diseases and conditions involving mitochondrial dysfunction, including changes in mtDNA copy number and/or concentration, such as, but not limited to, mtDNA depletion.

### BRIEF DESCRIPTION OF THE FIGURES

FIG. 1A represents an alignment of amino acid sequences of the polymerase domain of POLG1 protein from various species and shows that aspartic acid at position 1135 (with reference to the H. sapiens amino acid sequence) is evolutionarily conserved.

FIG. 1B shows a schematic of the development of inducible D1135A-POLG1 (mtDNA-depleter) transgenic mouse model. D1135A-POLG1-expressing mouse was created by microinjection of the pTRE-Tight-BI-AcGFP1-D1135-POLG1 construct into the one-cell stage egg from C57BL/6 mouse. The D1135A-POLG1-positive founder male mouse (Mouse I) was bred with the CAG-rtTA3 female mouse (Mouse II, Jackson Laboratories, stock #016532) resulting in the D1135A-POLG1 transgenic animal (Mouse III).

FIG. 1C shows pups genotyping revealing the presence of D1135A-POLG1, rtTA, and GFP.

FIG. 1D shows whole-body imaging confirming the expression of GFP in only mtDNA-depleter mice.

FIG. 1E shows RT-PCR analyses confirming dox-dependent expression of D1135A-POLG1 in only mtDNA-depleter mice

FIG. 2A shows quantification of mtDNA content (mean±s.e.m; *P<0.05, Student's t test) in skin samples from wild-type control (WT; n=3) and mtDNA-depleter (Depleter; n=3) mice after 2 months of continuous dox induction.

FIG. 2B shows RT-PCR analysis of mitochondrial genes and nuclear genome-encoded regulators of mitochondrial biogenesis (PGC1α) and mitochondrial transcription and genome regulation (TFAM) in the skin of wild-type control (WT; n=3) and mtDNA-depleter (Depleter; N=3) mice after 2 months of continuous dox induction.

FIG. 2C shows western blot analysis of OXPHOS subunits in the skin of wild-type control (WT; n=3) and mtDNA-depleter (Depleter; N=3) mice after 2 months of continuous dox induction.

FIG. 2D shows BN-PAGE analysis of OXPHOS super complexes in the skin of wild-type control (WT; n=3) and mtDNA-depleter (Depleter; N=3) mice after 2 months of continuous dox induction.

FIG. 2E shows enzymatic activities of OXPHOS complex I (mean s.e.m; *P<0.05, Student's t test) in the skin of wild-type control (WT; n=3) and mtDNA-depleter (Depleter; n=3) mice after 2 months of continuous dox induction.

FIG. 2F shows enzymatic activities of OXPHOS complex II (mean±s.e.m; *P<0.05, Student's t test) in the skin of wild-type control (WT; n=3) and mtDNA-depleter (Depleter; n=3) mice after 2 months of continuous dox induction.

FIG. 2G shows enzymatic activities of OXPHOS complex III (mean±s.e.m; *P<0.05, Student's t test) in the skin of wild-type control (WT; n=3) and mtDNA-depleter (Depleter; n=3) mice after 2 months of continuous dox induction.

FIG. 2H shows enzymatic activities of OXPHOS complex IV (mean±s.e.m; *P<0.05, Student's t test) in the skin of wild-type control (WT; n=3) and mtDNA-depleter (Depleter; n=3) mice after 2 months of continuous dox induction.

FIG. 2I shows enzymatic activities of OXPHOS complex V (mean±s.e.m; *P<0.05, Student's t test) in the skin of wild-type control (WT; n=3) and mtDNA-depleter (Depleter; n=3) mice after 2 months of continuous dox induction.

FIG. 2J shows quantification of mtDNA content (mean±s.e.m; *P<0.05, Student's t test) in heart, lung, brain and liver samples from wild-type control (WT; n=3) and mtDNA-depleter (Depleter; n=3) mice after 2 months of continuous dox induction.

FIG. 3A shows mtDNA-depleter mice develop skin wrinkles (panel ii), hair loss (panel ii), and kyphosis (panel iii) after 4-8 weeks of continuous dox-mediated induction of POLG1 as compared to wild-type control (panel i).

FIG. 3B shows quantitative assessment of body weight of mtDNA-depleter (n=30) and wild-type control mice (n=30) after 60 days of continuous dox induction of POLG1. Data are expressed as mean±s.e.m; *P<0.05, Student's t test.

FIG. 3C shows quantitative assessment of body length of mtDNA-depleter (n=30) and wild-type (WT) control mice (n=30) after 60 days of continuous dox induction of POLG1. Data are expressed as mean±s.e.m; *P<0.05, Student's t test.

FIG. 3D shows quantitative assessment of lean mass/length ratio of mtDNA-depleter (n=30) and wild-type (WT) control mice (n=30) after 60 days of continuous dox induction of POLG1. Data are expressed as mean±s.e.m; *P<0.05, Student's t test.

FIG. 3E shows quantitative assessment of hair loss phenotypic changes in mtDNA-depleter (n=30) and wild-type (WT) control mice (n=30) after 60 days of continuous dox induction of POLG1.

FIG. 3F shows quantitative assessment of wrinkled skin phenotypic changes in mtDNA-depleter (n=30) and wild-type (WT) control mice (n=30) after 60 days of continuous dox induction of POLG1.

FIG. 4A shows mtDNA-depleter mice demonstrate a very strong alopecia and wrinkled skin (panel i), kyphosis (panel ii), progeroid head (panel iii), and darkly pigmented ear pinnae (panel iv) phenotypic changes after 60 days of continuous dox induction of POLG1 as compared to wild-type mice.

FIG. 4B shows representative images of an mtDNA-depleter mouse after 60 days of continuous dox induction of POLG1 showing the gross phenotypic changes in the size and appearance compared to an age-matched wild-type control littermate.

FIG. 4C shows the patterns of hair loss in male mtDNA-depleter mice after 60 days of continuous dox induction of POLG1.

FIG. 4D shows the patterns of hair loss in female mtDNA-depleter mice after 60 days of continuous dox induction of POLG1.

FIG. 4E shows representative images illustrating gradual time-dependent phenotypic changes in skin wrinkles and hair loss in a female mtDNA-depleter mouse after continuous dox induction (panels i-iv)

FIG. 5 shows representative hematoxylin- and eosin-stained cross-sections of brain (cerebrum), liver, heart (myocardium), and lung from wild-type control (n=3) and mtDNA-depleter mice (n=3) after 60 days of continuous dox induction. Scale bar is 100 μM.

FIG. 6A shows representative hematoxylin- and eosin-stained sections of dorsal skin from wild-type control (n=3) (i and ii) and mtDNA-depleter mice (n=3) (iii-vi) after 2 months of continuous dox induction. While the skin of wild-type mice shows the presence of normal skin histology (i, ×10), the skin of mtDNA-depleter mice shows hyperplastic epidermis with hyperkeratosis (black arrow), dysfunctional hair follicles containing keratinaceous debris and/or malformed hair (yellow arrow), and increased the number of inflammatory cells in the dermis (arrowhead) (iii, ×10). Skin sections at higher magnification show the presence of normal telogen hair follicles (ii, ×40) in wild-type control mice and aberrant telogen (iv, ×40) and anagen hair follicles (vi, ×20) with defective sebaceous glands. Panel v shows ruptured follicular cyst surrounded by granulomatous and mixed inflammatory infiltrate in mtDNA-depleter mice.

FIG. 6B shows quantification of epidermal thickness (mean±s.e.m; *P<0.05, Student's t test) in skin samples from wild-type control (WT; n=3) and mtDNA-depleter (depleter; n=3) mice after 2 months of continuous dox induction.

FIG. 6C shows quantification of hair follicles in telogen stages of hair cycle (mean±s.e.m; *P<0.05, Student's t test) in skin samples from wild-type control (WT; n=3) and mtDNA-depleter (depleter; n=3) mice after 2 months of continuous dox induction.

FIG. 6D shows quantification of hair follicles in anagen stages of hair cycle (mean s.e.m; *P<0.05, Student's t test) in skin samples from wild-type control (WT; n=3) and mtDNA-depleter (depleter; n=3) mice after 2 months of continuous dox induction.

FIG. 6E shows quantification of epidermal proliferation (PCNA⁺) in skin samples from wild-type control (n=3) and mtDNA-depleter (n=3) mice after 2 months of continuous dox induction.

FIG. 6F shows representative images of PCNA immuno-stained cross-sections of skin from wild-type control (n=3) and mtDNA-depleter mice (n=3) after 2 months of dox induction. The basement membrane position in these images is marked with dotted lines.

FIG. 6G shows representative electron micrographs of skin samples from wild-type control (n=3) and mtDNA-depleter mice (n=3) after 2 months of dox induction. Skin from mtDNA-depleter mice revealed a severely disturbed mitochondrial structure with loss of cristae and degeneration of intramitochondrial structures.

FIG. 7A shows immunocytochemical and histochemical analyses of skin sections demonstrating the presence of increased number of inflammatory cells including mast cells (Giemsa stain-positive cells), granulocytes (MPO-positive cells), macrophages and histiocytes (CD163-positive cells), and B lymphocytes (Pax-5-positive cells) in the dermis, as well as in perifollicular and periepidermal location of mtDNA-depleter mice after 2 months of continuous dox-induction. The skin sections of wild-type mice are predominantly negative for MPO, CD163, and Pax-5 staining. Arrows indicate the presence of inflammatory cells in the skin sections.

FIG. 7B shows quantitative analysis of Giemsa-positive mast cells in the skin sections of wild-type control (WT; n=3) and mtDNA-depleter (Depleter; n=3) mice (mean±s.e.m; *P<0.05, Student's t test).

FIG. 7C shows RT-PCR analysis of inflammatory genes in the skin RNA samples of wild-type control (WT; n=3) and mtDNA-depleter mice (Depleter; n=3) after 2 months of continuous dox induction.

FIG. 7D shows RT-PCR analysis of genes involved in maintenance of extracellular matrix and inflammation in the skin RNA samples of wild-type control (WT; n=3) and mtDNA-depleter mice (Depleter; n=3) after 2 months of continuous dox induction.

FIG. 8 shows representative images illustrating mRNA expression analyses of IGF1R, VEGF, MRPS5, and Klotho genes (marker genes of intrinsic aging) by RT-PCR in the skin samples of wild-type control (WT; n=3) and mtDNA-depleter mice (Depleter; n=3) after 2 months of dox induction.

FIG. 9A shows representative images of an mtDNA-depleter mouse showing skin wrinkles and hair loss after 2 months of continuous dox induction (+dox; mtDNA depletion) (ii; n=3) and reversal of these phenotypic changes after 1 month of dox withdrawal (−dox; mtDNA repletion) (iii; n=3). Wild-type control mice (i; n=3) did not show any change in skin phenotype after dox treatment or after 1 month of dox withdrawal.

FIG. 9B shows a representative hematoxylin- and eosin-stained sections of dorsal skin from wild-type control mice (i; n=3), mtDNA-depleter mice after 2 months of continuous dox induction (mtDNA depletion) (ii; n=3), and mtDNA-depleter mice after 1 month of dox withdrawal (−dox; mtDNA repletion) (iii; n=3).

FIG. 9C shows a representative giemsa staining of skin sections from wild-type control mice (i; n=3), mtDNA-depleter mice after 2 months of continuous dox induction (+dox; mtDNA depletion) (ii; n=3), and mtDNA-depleter mice after 1 month of dox withdrawal (−dox; mtDNA repletion) (iii; n=3) showing the presence of increased number of mast cells in the dermis and perifollicular as well as near-epidermal locations of mtDNA-depleter mice compared with skin sections of wild-type mice. Compared with mtDNA-depleter mice, the lower number of mast cells is present in the skin of mtDNA-depleter mice after 2 months of continuous dox induction followed by 1 month of dox withdrawal. Arrows indicate the presence of Giemsa-positive cells in the skin sections.

FIG. 9D shows quantification of epidermal thickness (mean±s.e.m; *P<0.05, Student's t test) in skin samples from wild-type control (WT; n=3) and mtDNA-depleter (Depletion; n=3) mice after 2 months of continuous dox induction and after 2 months of continuous dox induction followed by 1 month of dox withdrawal (Repletion).

FIG. 9E shows quantification of hair follicles in telogen stage of hair cycle (mean±s.e.m; *P<0.05, Student's t test) in skin samples from wild-type control (WT; n=3) and mtDNA-depleter (Depletion; n=3) mice after 2 months of continuous dox induction and after 1 month of dox withdrawal (Repletion).

FIG. 9F shows quantification of hair follicles in anagen stage of hair cycle (mean±s.e.m; *P<0.05, Student's t test) in skin samples from wild-type control (WT; n=3) and mtDNA-depleter (Depletion; n=3) mice after 2 months of continuous dox induction and after 2 months of continuous dox induction followed by 1 month of dox withdrawal (Repletion).

FIG. 9G shows quantitative analysis of Giemsa-positive mast cells (mean±s.e.m; *P<0.05, Student's t test) in the skin sections of wild-type control (WT; n=3) and mtDNA-depleter (Depletion; n=3) mice after 2 months of continuous dox induction and after 2 months of continuous dox induction followed by 1 month of dox withdrawal (Repletion).

FIG. 9H shows quantification of mtDNA content (mean±s.e.m; *P<0.05, Student's t test) in skin samples from wild-type control (WT; n=3) and mtDNA-depleter (Depletion; n=3) mice after 2 months of continuous dox induction and after 1 month of dox withdrawal (Repletion).

FIG. 9I shows representative gel images illustrating RT-PCR analysis of the PGC1α, TFAM, COXII, NDI, and RNU6B genes in the skin RNA samples of wild-type control (n=3) and mtDNA-depleter mice (n=3) after 2 months of continuous dox induction followed by 1 month of dox withdrawal (Repletion).

FIG. 9J shows representative gel images illustrating RT-PCR analysis of the NFλB, cyclooxygenase 2, MMP2, MMP2, MMP9, TIMP1, and RNU6B genes in the skin RNA samples of wild-type control (n=3) and mtDNA-depleter mice (n=3) after 2 months of continuous dox induction followed by 1 month of dox withdrawal (Repletion).

FIG. 10A shows representative gels providing RT-PCR analysis of genes involved in maintenance of extracellular matrix and inflammation from skin fibroblasts generated from mtDNA-depleter mice without dox induction of POLG1 and after dox (1 μg/ml) induction of POLG1.

FIG. 10B shows increased proliferation of skin fibroblasts generated from mtDNA-depleter mice without (control) and with (D1135A-POLG1) dox (1 μg/ml) induction of POLG1.

FIG. 11 shows the amino acid sequence of the human POLG1 protein.

### SUMMARY OF THE INVENTION

mtDNA depletion is involved in mtDNA depletion syndromes, mitochondrial diseases, aging and aging-associated chronic diseases, and other human pathologies. To evaluate the consequences of mitochondrial dysfunction, the present disclosure provides an animal model which allows for the inducible and controlled depletion of mtDNA (i.e., an inducible mtDNA-depleter animal).

In a first aspect, the present disclosure provides an inducible mtDNA-depleter animal model comprising a mutant DNA polymerase subunit gamma-1 (POLG1) polypeptide to induce depletion of mtDNA in the whole animal or in various cells/tissues when the mutant POLG1 polypeptide is expressed.

In a second aspect, the present disclosure provides an inducible mtDNA-depleter animal model comprising an inducible construct comprising an inducible expression control sequence and a nucleic acid sequence encoding the mutant POLG1 polypeptide.

In a third aspect, the present disclosure provides an inducible mtDNA-depleter animal model comprising an inducible construct comprising an inducible expression control sequence, a nucleic acid sequence encoding the mutant POLG1 polypeptide, and a nucleic acid sequence encoding a transactivator polypeptide.

In a fourth aspect, the present disclosure provides an inducible mtDNA-depleter animal model comprising an inducible construct comprising an inducible expression control sequence and a nucleic acid sequence encoding the mutant POLG1 polypeptide, and a second construct comprising a second expression control sequence and a nucleic acid sequence encoding a transactivator polypeptide.

In a fifth aspect, the present disclosure provides an inducible mtDNA-depleter animal model of any of the foregoing aspects, wherein the inducible construct is the pTRE-Tight-BI-AcGFP1 construct.

In a sixth aspect, the present disclosure provides an inducible mtDNA-depleter animal model of any of the foregoing aspects, wherein the inducible construct comprises a tetracycline responsive element (TRE) and a promoter and the transactivator polypeptide is a tetracycline response transactivator.

In a seventh aspect, the present disclosure provides an inducible mtDNA-depleter animal model of any of the first to sixth aspects, wherein the mutant POLG1 polypeptide is expressed in the presence of the inducer and not expressed to an appreciable degree or at all in the absence of the inducer compound.

In an eighth aspect, the present disclosure provides an inducible mtDNA-depleter animal model of any of the first to sixth aspects, wherein the mutant POLG1 polypeptide is expressed in the absence of the inducer and not expressed to an appreciable degree or at all in the presence of the inducer compound.

In a ninth aspect, the present disclosure provides an inducible mtDNA-depleter animal model of any of the foregoing aspects, wherein the expression of the mutant POLG1 polypeptide occurs in a selected cell or tissue of the mtDNA-depleter animal.

In a tenth aspect, the present disclosure provides an inducible mtDNA-depleter animal model of any of the foregoing aspects, wherein the expression of the mutant POLG1 polypeptide occurs in all or substantially all (for example, greater than 95%) cells of the mtDNA-depleter animal.

In an eleventh aspect, the present disclosure provides an inducible mtDNA-depleter animal model of any of the foregoing aspects, wherein the mutation in the POLG1 polypeptide is a dominant negative mutation, for example, an aspartic acid to alanine amino acid change at the evolutionarily conserved site in the polymerase domain of POLG1 at 1135 position of SEQ ID NO: 45 (D1135A-POLG1) or at a corresponding aspartic acid residue.

In a twelfth aspect, the present disclosure provides an inducible mtDNA-depleter animal model of any of the foregoing aspects, wherein the expression of the mutant POLG1 polypeptide can be regulated to provide for severe, moderate, or low levels of mitochondria dysfunction by increasing or decreasing the doses of the inducer compound given to the mouse.

In a thirteenth aspect, the present disclosure provides an inducible mtDNA-depleter animal model of any of the foregoing aspects, wherein the inducer compound is tetracycline or doxycycline.

In a fourteenth aspect, a cell or tissue of any of the foregoing aspects is isolated from the mouse and used as a component of an in vitro or in vivo test system.

In a fifteenth aspect, any of the foregoing aspects may be used to identify a compound for the treatment of a disease or condition, such as, but not limited to, mtDNA depletion syndromes, skin wrinkles, hair loss, increased epidermal thickness, epidermal hyperplasia, acanthosis, hyperkeratosis, cardiovascular disease, diabetes, cancer, aging, and aging-associated chronic diseases or pathologies, such as, but not limited to age-associated neurological disorders.

As discussed in more detail herein, mice expressing a mutant POLG1 showed reduced mtDNA content, reduced mtDNA concentration, reduced mitochondrial gene expression, and instability of super complexes involved in oxidative phosphorylation (OXPHOS) resulting in reduced OXPHOS enzymatic activities. The present disclosure shows that ubiquitous depletion of mtDNA in this animal model leads to predominant and profound effects on the skin resulting in skin wrinkles and visual hair loss with an increased number of dysfunctional hair follicles and inflammatory responses. Development of skin wrinkle was associated with the significant epidermal hyperplasia, hyperkeratosis, increased expression of matrix metalloproteinases, and decreased expression of matrix metalloproteinase inhibitor TIMP1. The present disclosure also shows that markedly increased skin inflammation is a contributing factor in skin pathology. Histopathologic analyses revealed dysfunctional hair follicles. Animals expressing the mutant POLG1 (mtDNA-depleter) also show changes in expression of aging-associated markers including IGF1R, KLOTHO, VEGF, and MRPS5. mtDNA-depleter animals in which the expression of the mutant POLG1 was inhibited (for example, by removing the inducer compound) (mtDNA repleter animals) showed that, mitochondrial function, as well as the skin and hair pathology, is reversible. To the knowledge of the inventors, the demonstration that inhibition of mitochondrial function (for example, depletion of mtDNA) can lead to skin and hair pathologies and that restoration of mitochondrial functions (for example, restoration of mtDNA) can reverse the skin and hair pathology is unprecedented.

These and other features and advantages of the present disclosure will be set forth or will become more fully apparent in the description that follows and in the appended claims. The features and advantages may be realized and obtained by means of the instruments and combinations particularly pointed out herein and in the appended claims. Furthermore, the features and advantages of the present disclosure may be learned by the practice of the methods and/or use of the compounds and compositions described herein or will be apparent from the description in the present disclosure.

### Definitions

As used herein, the term “corresponding aspartic acid” means an aspartic acid (D) residue that is mutated to an alanine (A) residue in a POLG1 amino acid sequence that is the equivalent of the aspartic acid at position 1135 of the human POLG1 sequence (SEQ ID NO: 45). In a particular embodiment, the “corresponding aspartic acid” is flanked on the amino terminus side by an amino acid sequence of S/T I/V H X, I S/T I/V H X, or C/A I S/T I/V H X, and/or on the carboxy terminus side by an amino acid sequence of X E V/I R, X E V/I R Y/F, or X E V/I R Y/F L, wherein “X” indicates the aspartic acid amino acid that is mutated or will be mutated to alanine.

As used herein, the terms “depleted,” “depletion,” or “depleter” with respect to mtDNA refers to a decrease in mtDNA copy number and/or concentration in a transgenic non-human animal, tissue, or cell of the present disclosure which is caused by the expression of a mutant POLG1 polypeptide, as determined by a comparison to a control non-human animal, tissue, or cell (for example, a transgenic non-human animal that has not expressed the mutant POLG1 polypeptide).

As used herein, the terms “mutant POLG1” or “mutated POLG1” refers to a POLG1 amino acid sequence from a particular species that contains at least one mutation as compared to the wild-type POLG1 sequence from that species. A mutation need not cause a disease. A single mutation or more than one mutation may be present. In a particular embodiment, a single dominant negative mutation may be present, optionally with one or additional mutations.

As used herein, the term “nucleic acid sequence” includes sequence of DNA, RNA, and nucleic acid analogs, and nucleic acid sequence that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

As used herein, the term “operatively linked” or “operably linked” refers to the connection of elements being a part of a functional unit such as a gene or an open reading frame (e.g., encoding a mutant POLG1). Accordingly, by operatively linking an expression control sequence (for example, a promoter) to a heterologous nucleic acid sequence (for example, a transgene) encoding a polypeptide, the expression control sequence and the nucleic acid sequence become part of the functional unit encoding a protein comprising the amino acid sequence encoded by the heterologous nucleic acid sequence. The linking of the expression control sequence to the nucleic acid sequence enables the transcription of the nucleic acid sequence under the control of the expression control sequence. By operatively linking an expression control sequence and two heterologous nucleic acid sequences (for example, 2 transgenes) each encoding a polypeptide, the expression control sequence and the two heterologous nucleic acid sequences becomes part of the functional unit encoding proteins comprising the amino acid sequences encoded by the two heterologous nucleic acid sequences. By operatively linking two heterologous nucleic acid sequences to an expression control sequence, the two heterologous nucleic acid sequences can be co-expressed.

As used herein, the terms “repleted,” “repletion,” or “repleter” with respect to mtDNA refers to a increase in mtDNA copy number and/or concentration in a non-human animal, tissue, or cell of the present disclosure which is caused by ceasing the expression of a mutant POLG1 polypeptide, as determined by a comparison to a mtDNA copy number and/or concentration immediately before repletion. In certain aspects, mtDNA is repletion results in mtDNA copy number and/or concentration approximately equal to the mtDNA copy number and/or concentration observed a control non-human animal, tissue, or cell (for example, a transgenic non-human animal that has not expressed the mutant POLG1 polypeptide).

As used herein, the term “tetracycline derivative” refers to a tetracycline related compound that is capable of binding to a tetracycline response element. A preferred tetracycline derivative is doxycycline (dox).

As used herein, the term “tetracycline responsive” means that the element of polypeptide exhibits a specific function or a change in function in the presence of tetracycline or a tetracycline derivative. For example, a tetracycline response transactivator is an activator that exhibits a change in function in the presence of tetracycline or a tetracycline derivative.

As used herein, the term “tetracycline responsive transactivator” or “TA” means a polypeptide that is capable of binding to a polypeptide and/or nucleic acid sequence to stimulate transcription of a nucleic acid sequence downstream from a tetracycline response element.

As used herein, the term “transgene” is used herein to describe genetic material that has been or is about to be artificially introduced into a genome of a host organism (e.g., a non-human animal) and that is transmitted to the progeny of that host organism. The transgene will typically comprise a nucleic acid sequence that contains non-coding and/or coding sequences that usually, but not necessarily, impart or elicit an activity (e.g., regulation of transcription of a nucleic acid sequence, production of a nucleic acid sequence including a coding and/or non-coding sequence, etc.). In some embodiments, the transgene comprises a nucleic acid sequence that is capable of being transcribed into RNA and optionally translated and/or expressed into a polypeptide under appropriate conditions. In some embodiments, the transgene comprises a nucleic acid sequence encoding a polypeptide. In some embodiments, the transgene comprises a targeting cassette for introducing a genetic modification into a genome. Any of various methods can be used to introduce a transgene into a non-human animal to produce a transgenic non-human animal. Such techniques are well-known in the art and include, but are not limited to, pronuclear microinjection, viral infection and transformation of embryonic stem cells and IPS cells. Methods for generating transgenic animals that can be used include, but are not limited to, those described in J. P. Sundberg and T. Ichiki, Eds., Genetically Engineered Mice Handbook, CRC Press; 2006; M. H. Hofker and I. van Deursen, Eds., Transgenic Mouse Methods and Protocols, Humana Press, 2002; A. L. Joyner, Gene Targeting: A Practical Approach, Oxford University Press, 2000; Manipulating the Mouse Embryo: A Laboratory Manual, 3rd edition, Cold Spring Harbor Laboratory Press; 2002, ISBN-10: 0879695919; K. Turksen (Ed.), Embryonic stem cells: methods and protocols in Methods Mol, Biol. 2002; 185, Humana Press; Current Protocols in Stem Cell Biology, ISBN: 978047015180; Meyer et al. PNAS USA, vol. 107 (34), 15022-15026.

As used herein, the term “transgenic” is used broadly herein and refers to a genetically modified host organism whose genetic material has been altered using genetic engineering techniques. For example, a “transgenic” non-human animal refers to an animal which comprises a genetic modification, which has been introduced into the genome of the non-human animal. The term has a similar meaning with respect to a part of the host organism, a tissue from the host organism, or a cell of the host organism.

### Background

Mitochondrial dysfunction is associated with many mitochondrial diseases, most of which are the result of dysfunctional mitochondrial OXPHOS. Mitochondrial OXPHOS accounts for the generation of most of the cellular adenosine triphosphate (ATP) in a cell. The OXPHOS function largely depends on the coordinated expression of the proteins encoded by both nuclear and mitochondrial genomes. The human mitochondrial genome encodes for 13 polypeptides of the OXPHOS system, and the nuclear genome encodes the remaining more than 85 polypeptides required for the assembly of OXPHOS system. Mitochondrial DNA (mtDNA) depletion impairs OXPHOS and leads to mtDNA depletion syndromes (MDSs) (Alberio, et al., Mitochondrion 7, 6-12, 2007; Ryan, M et al., Annu. Rev. Biochem. 76, 701-722, 2007). The MDSs are a heterogeneous group of disorders, characterized by low mtDNA levels in specific tissues. In different target organs, mtDNA depletion leads to specific pathological changes (Tuppen, et al., Biochim. Biophys. Acta 1797, 113-128, 201)). MDSs result from the genetic defects in the nuclear-encoded genes that participate in mtDNA replication, and mitochondrial nucleotide metabolism and nucleotide salvage pathway (Alberio, et al., Mitochondrion 7, 6-12, 2007). mtDNA depletion is also implicated in other human diseases such as mitochondrial diseases, cardiovascular, diabetes, age-associated neurological disorders, and cancer.

A general decline in mitochondrial function has been extensively reported during aging. Furthermore, mitochondrial dysfunction is known to be a driving force underlying age-related human diseases. A mouse that carries elevated mtDNA mutation is also shown to present signs of premature aging. In addition to mutations in mtDNA, studies also suggest a decrease in mtDNA content and mitochondrial number with age. Notably, there is an age-related mtDNA depletion in a number of tissues. mtDNA depletion is also frequently observed among women with premature ovarian aging. Low mtDNA copy number is linked to frailty and, for a multiethnic population, is a predictor of all-cause mortality. A recent study revealed that humans on an average lose about four copies of mtDNA every ten years. This study also identified an association of decrease in mtDNA copy number with age-related physiological parameters.

To help define the role of mtDNA depletion and repletion in aging and various diseases, an inducible transgenic non-human animal model expressing a mutated POLG1 polypeptide (such as, but not limited to, a POLG1 polypeptide expressing a dominant-negative (DN) mutation) that induces mitochondrial dysfunction (for example by depletion of mtDNA) in the whole animal or selected cells/tissues is provided. Interestingly, skin wrinkles and visual hair loss were among the earliest and most predominant phenotypic changes observed in these mice. In the present study, mtDNA depletion-induced phenotypic changes were shown to be reversible by restoration of mitochondrial function upon repletion of mtDNA.

Accumulating evidence suggests a strong link between mitochondrial dysfunction, mitochondrial diseases, aging, and aging-associated diseases. Notably, increased somatic mtDNA mutations and decline in mitochondrial functions have been extensively reported during human aging. Studies also suggest a decrease in mtDNA content and mitochondrial number with age.

The present disclosure shows that the depletion of mtDNA predominantly leads to a variety of physiological and phenotypic changes in the transgenic non-human animal, including, but not limited to, wrinkled skin and hair loss accompanied by an inflammatory phenotype, changes in mitochondrial protein expression, reduced expression of mitochondrial oxidative phosphorylation complexes, reduced stability of mitochondrial oxidative phosphorylation complexes, skin wrinkles, hair loss, increased epidermal thickness, epidermal hyperplasia, acanthosis, hyperkeratosis, increased expression of at least one gene selected from the group consisting of: NF-κB, COX-2, INF-β1, CCL5, MMP1, MMP2, MMP9, MMP13, IGF1R, VEGF, and MRPS5, decreased expression of TIMP1 and KLOTHO, increased skin inflammation, and aberrant hair follicles. Wrinkled skin and hair loss are obvious features of skin aging and aging-associated phenotypic changes in humans. The present disclosure also demonstrates that these aging-associated phenotypic changes can be reversed by repletion of mtDNA content. To the knowledge of the inventors, the foregoing have not been previously reported in the art.

Skin wrinkles are a hallmark of both intrinsic and extrinsic aging of the skin in humans. Mitochondrial dysfunction is implicated in both intrinsic and extrinsic aging. The presence of skin wrinkles, acanthosis, epidermal hyperplasia with hyperkeratosis, and marked inflammatory infiltrate in the skin of mtDNA-depleter mice) represent characteristics similar to the extrinsic aging of skin in humans. Furthermore, the changes in expression of intrinsic aging-associated genetic markers support intrinsic mechanisms underlying the phenotypic changes observed in mtDNA-depleter mice.

Loss of collagen fibers is reported to underlie skin wrinkles. A tight balance between the proteolytic matrix metalloprotease (MMP) enzymes and their tissue-specific inhibitor tissue inhibitor metalloproteinase-1 (TIMP1) is essential to maintain the collagen fiber content in the skin. Expression of MMPs is altered in the aged skin. Consistent with these reports, the skin of mtDNA-depleter mice showed increased expression of MMPs and decreased expression of TIMP1, indicating loss of balance contributing to the development of skin wrinkles. Repletion of mtDNA content restored MMP expression leading to a reversal of wrinkled skin and hair loss. These experiments show that mitochondria are regulators of skin aging and hair loss. This observation is surprising and suggests that epigenetic mechanisms underlying mitochondria-to-nucleus cross-talk must play an important role in the restoration of normal skin and hair phenotype.

mtDNA stress triggers inflammatory response. Inflammation also underlies aging and age-related diseases. Increased levels of markers of inflammation in the mtDNA-depleter mice indicate an activated immune response in the skin of mtDNA-depleter mice. Increased expression of NF-κB, a master regulator of the inflammatory response, upon mtDNA depletion and its reduced expression after the restoration of mtDNA content suggests that NF-κB signaling is a critical mechanism contributing to the skin and hair follicle pathologies observed in mtDNA-depleter mice. Furthermore, a unique feature of proteins encoded by mtDNA is N-formyl-methionine at the N terminus. N-formylated peptides when present in the extracellular space are known to act as mitochondrial damage-associated molecular patterns and activate neutrophils or activate keratinocyte-intrinsic responses resulting in the recruitment of immune cells. While previous studies have shown increased inflammatory responses associated with disruption of mtDNA homeostasis, the prior studies relied on a targeted approach. The results of the present disclosure can be differentiated from these prior studies in a number of ways. First, the results of the present disclosure were generated using a whole-animal approach to ubiquitously deplete mtDNA to disrupt mitochondrial function instead of a targeted approach in the epidermis. Thus, the present disclosure indicates an important role of mitochondria in the skin when compared to other tissues. Secondly, the present disclosure demonstrates that reversal of inflammatory gene expression strongly suggest a role for epigenetics in the regulation of mitochondrial genes and mitochondrial function. Lastly, the present disclosure demonstrates clearance of infiltrated immune cells from dermis upon restoration of the mitochondrial function, a finding not observed in the prior art. Also, the short lifespan of prior art animal models prevented any aging study. However, the present disclosure shows the development of wrinkles and loss of hair, a persistent and profound feature of human aging. Similarly, epidermis-specific knockout mouse shows defects in epidermal differentiation and hair follicle morphogenesis during embryonic development. However, due to a short life of these knockout mouse, these studies did not observe the effect of mitochondrial dysfunction leading to skin wrinkles and hair loss in adult mice.

In summary, development of the transgenic non-human animal model disclosed demonstrates that the loss of mtDNA homeostasis is responsible for the development of skin wrinkles, hair loss (for example, due to abnormal development of adnexal structures) and other pathologies described herein. The disclosed transgenic non-human animal model allows for the ubiquitous suppression and restoration of mitochondrial function in the whole animal or in specific cells/tissues. The transgenic non-human animal model disclosed can be used to rapidly identify genes and pathways involved in the pathogenesis and amelioration of mtDNA diseases. Furthermore, the transgenic non-human animal model disclosed can be used to generate tissue-specific modulation of mitochondrial functions to determine, for various organs, the effects of mitochondria on in vivo aging, and pathogenesis of MDS and other mitochondrial diseases. Furthermore, the transgenic non-human animal model disclosed provides a unique screening platform for the identification and development of therapeutic compounds for the prevention and/or treatment of mitochondrial diseases. For example, the transgenic non-human animal model disclosed can be used for the identification and development of therapeutics to augment mitochondrial function for the treatment of aging-associated pathology, such as, but not limited to skin wrinkles, hair loss, and other skin and hair pathology, and other human diseases in which mitochondrial dysfunction plays a significant role.

### Methods

The present application shows that the depletion of mtDNA predominantly leads to wrinkled skin and hair loss accompanied by an inflammatory phenotype. Wrinkled skin and hair loss are obvious features of skin aging and aging-associated phenotypic changes in humans. The present application also demonstrates that these aging-associated phenotypic changes could be reversed by repletion of mtDNA content. To the knowledge of the inventors, the foregoing have not been previously reported in the art.

The present disclosure provides a transgenic non-human animal model that contain a nucleic acid sequence encoding a mutant POLG1 polypeptide and that express the mutant POLG1 polypeptide in a controlled manner. Through the use of the transgenic non-human animal model described herein, the functioning of the mitochondrial pathway may be studied and deciphered. For example, upon expression of the mutant POLG1 polypeptide, mtDNA is depleted. When the mutant POLG1 polypeptide is no longer expressed, mtDNA is repleted. Embryos, cells, and tissues from such transgenic non-human animal are also provided. The present disclosure further provides constructs for making the transgenic non-human animal model that is capable of expressing a mutant POLG1 polypeptide and methods of making the same. Methods of using the transgenic non-human animal model expressing a mutant POLG1 polypeptide to study various aspects of human disease and to identify and develop therapeutics for the treatment of various human diseases are also provided.

In one embodiment, the present disclosure provides for a method to create mtDNA depletion and repletion in the whole animal in any developmental stages of an animal.

In another embodiment, the present disclosure provides for a method to create mtDNA depletion and repletion in any desired tissue or organ in any developmental stages of an animal.

In another embodiment, the present disclosure provides for a method to create mtDNA depletion and repletion in any desired cell types in any developmental stages of an animal.

In another embodiment, the present disclosure provides for a method to create mitochondrial dysfunction and restore mitochondrial function in the whole animal in any developmental stages of an animal.

In another embodiment, the present disclosure provides for a method to create mitochondrial dysfunction and restore mitochondrial function in any desired tissue or organ in any developmental stages of an animal.

In another embodiment, the present disclosure provides for a method to create mitochondrial dysfunction and restore mitochondrial function in any desired cell types in any developmental stages of an animal.

In another embodiment, the present disclosure provides for a method to determine the genes and gene network involved in mtDNA depletion, repletion and dysfunction at in any developmental stages of an animal.

In another embodiment, the present disclosure provides for a method to induce or reverse mitochondrial dysfunction by targeting a gene and gene network involved in mtDNA depletion, repletion and dysfunction and restoration at in any developmental stages of an animal.

In another embodiment, the present disclosure provides for a method to determine the metabolic pathways and networks involved in mtDNA depletion, repletion and dysfunction, and restoration at in any developmental stages of an animal.

In another embodiment, the present disclosure provides for a method to induce or reverse mitochondrial dysfunction by targeting a metabolic pathway or metabolic network involved in mtDNA depletion, repletion and dysfunction and restoration at in any developmental stages of an animal.

In another embodiment, the present disclosure provides for a method for identification of secretary exosomes, protein, and miRNA involved in mtDNA depletion, repletion and dysfunction, and restoration at in any developmental stages of an animal.

In another embodiment, the present disclosure provides for a method to induce or reverse mitochondrial dysfunction by targeting a secretary exosome, protein, and/or miRNA involved in mtDNA depletion, repletion and dysfunction and restoration at in any developmental stages of an animal.

In another embodiment, the present disclosure provides for a method of induction and reversal of skin wrinkles upon mtDNA depletion, repletion and dysfunction, and restoration

In another embodiment, the present disclosure provides for a method of induction and reversal of hair loss upon mtDNA depletion, repletion and dysfunction, and restoration.

In another embodiment, the present disclosure provides for a method of induction and reversal of gene network involved in skin wrinkles upon mtDNA depletion, repletion and dysfunction, and restoration.

In another embodiment, the present disclosure provides for a method of induction and reversal of gene network involved hair loss upon mtDNA depletion, repletion and dysfunction, and restoration.

In another embodiment, the present disclosure provides for a method of induction and reversal of skin and gut microbiome involved in skin wrinkles upon mtDNA depletion, repletion and dysfunction, and restoration.

In another embodiment, the present disclosure provides for identification of metabolites involved in induction and reversal of hair loss upon mtDNA depletion, repletion and dysfunction, and restoration

In another embodiment, the present disclosure provides for identification of agents that prevent a disease or condition due, at least in part, to mitochodrial dysfunction.

In another embodiment, the present disclosure provides for identification of agents that prevent or treat hair loss due, at least in part, to mitochodrial dysfunction.

In another embodiment, the present disclosure provides for identification of agents that prevent or treat skin wrinkles due, at least in part, to mitochodrial dysfunction.

In another embodiment, the present disclosure provides for a therapeutic composition containing a preventative or therapeutic agent, or metabolite thereof, to prevent or treat a disease or condition due, at least in part, to mitochondrial dysfunction.

In another embodiment, the present disclosure provides for a therapeutic composition containing a preventative or therapeutic agent, or metabolite thereof, to prevent or treat skin wrinkles due, at least in part, to mitochondrial dysfunction.

In another embodiment, the present disclosure provides for a therapeutic composition containing a preventative or therapeutic agent, or metabolite thereof, to prevent or treat hair loss due, at least in part, to mitochondrial dysfunction.

In another embodiment, the present disclosure provides for methods of mitochondrial transfer or transplantation to prevent or treat a disease or condition due, at least in part, to mitochondrial dysfunction.

In another embodiment, the present disclosure provides for methods of mitochondrial transfer or transplantation to prevent or treat hair loss due, at least in part, to mitochondrial dysfunction.

In another embodiment, the present disclosure provides for methods of mitochondrial transfer or transplantation to prevent or treat skin wrinkles due, at least in part, to mitochondrial dysfunction.

In another embodiment, the present disclosure provides for an isolated cell from any of the animal models described herein, wherein the cell is used as a component of an in vitro or in vivo test system.

In another embodiment, the present disclosure provides for an isolated tissue from any of the animal models described herein, wherein the tissue is used as a component of an in vitro or in vivo test system.

In another embodiment, the present disclosure provides a therapeutic composition containing a preventative or therapeutic agent, or metabolite thereof, identified by a screening assay containing an isolated cell or tissue from any of the animal models described herein.

### Vectors and Nucleic Acids

In carrying out the methods of the present disclosure, a variety of nucleic acids may be introduced into the transgenic non-human animal to obtain expression of a polypeptide of interest (for example, a mutant POLG1 polypeptide), or for other purposes. A nucleic acid sequence of the disclosure is preferably operably linked to an expression control sequence, such as, but not limited to, a promoter. The expression control sequences can be from mice, human, or can be from other species. A nucleic acid sequence of the disclosure may contain multiple expression control sequences, such as, but not limited to, a polyadenylation sequence, a translation control sequence (e.g., an internal ribosome entry segment, IRES), an enhancer, an insulator sequence, a Kozak sequence, an inducible expression sequence, or an intron. Such additional expression control sequences may not be necessary, although they may increase expression by affecting transcription, stability of the mRNA, translational efficiency, or the like. Desired expression control sequences can be included in a nucleic acid sequence as desired to obtain optimal expression of the nucleic acids in the cell(s) of interest. Sufficient expression, however, can be obtained without such additional elements. In a preferred embodiment, a suitable expression control sequence is a promoter alone. In another preferred embodiment, a suitable expression control sequence is a transactivator response element, such as but not limited to, a TRE, and a promoter.

In some embodiments, a nucleic acid sequence encodes a polypeptide (either with or without a signal peptide). A signal peptide can be used such that an encoded polypeptide is directed to a particular cellular location (e.g., the cell surface). A nucleic acid sequence may further comprise a reporter to confirm the polypeptide of interest is being expressed. Non-limiting examples of reports include, but are not limited to, puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), xanthine-guanine phosphoribosyltransferase (XGPRT), and fluorescent polypeptides, such as, but not limited to, green fluorescent protein or yellow fluorescent protein. Preferably, the reporter is expressed only in conjunction with the polypeptide of interest (for example, a mutant POLG1).

A nucleic acid sequence may further comprise a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

In some embodiments, a nucleic acid sequence may contain an inhibitor sequence to prevent expression of a polypeptide encoded by a nucleic acid sequence. In preferred embodiments, the inhibitor sequence is flanked by recognition sequences for a recombinase such as, but not limited to, Cre or flippase (Flp). For example, the inhibitor sequence can be flanked by loxP recognition sites (34-bp recognition sites recognized by the Cre recombinase) or Flp recombinase target (FRT) recognition sites recognized by the Flp recombinase such that the inhibitor sequence can be excised from the nucleic acid sequence when desired. Such an approach may be used to achieve tissue specific or developmental specific expression of a polypeptide of interest. For example, a first non-human animal may contain a transgene comprising (in a 5′ to 3′ direction) a first expression control sequence (for example, a tissue specific promoter) for driving expression of a transactivator (for example, a tetracycline-responsive transactivator), an inhibitor sequence flanked by loxP recognition sites, a nucleic acid sequence encoding the transactivator, a second expression control sequence, preferably an inducible expression control sequence, comprising a transactivator responsive element (for example, a TRE and a promoter) for driving expression of a polypeptide of interest, and a nucleic acid sequence encoding the polypeptide of interest (for example, a mutant POLG1) operably linked to the second expression control sequence. In this example, the first expression control sequence is not operably-linked to the nucleic acid sequence encoding the transactivator due to the presence of the inhibitor sequence. As such, the transactivator is not expressed and the polypeptide of interest is not expressed. Activation of the polypeptide of interest (including tissue or temporal specific expression) can be accomplished, for example, by crossing the first transgenic non-human animal with a second transgenic non-human animal that ubiquitously expresses a Cre recombinase. Controlled excision of the inhibitor sequence operably-links the first expression control sequence and the nucleic acid sequence encoding the transactivator, allowing expression of the transactivator, which in turn binds the second expression control sequence and drives expression of the nucleic acid sequence encoding the polypeptide of interest.

Nucleic acid constructs (such as a transgene) can be introduced into embryonic, fetal, or adult artiodactyl/livestock cells of any type, including, for example, germ cells such as an oocyte or an egg, a progenitor cell, an adult or embryonic stem cell, or a primordial germ cell, using a variety of techniques. Non-limiting examples of techniques include the use of vectors, transposon systems, liposomes or other non-viral methods such as electroporation, microinjection, or calcium phosphate precipitation, that are capable of delivering nucleic acids to cells.

Nucleic acid sequences of the disclosure can be incorporated into vectors. A vector is a broad term that includes any specific DNA segment that is designed to move from a carrier into a target nucleic acid. A vector may be referred to as an expression vector, or a vector system, which is a set of components needed to bring about insertion of a desired nucleic acid sequence into a genome or other targeted sequence. In one embodiment, vector systems, such as viral vectors (e.g., retroviruses, adeno-associated virus and integrating phage viruses), and non-viral vectors (e.g., transposons) used for gene delivery in animals comprise: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

### Inducible Systems

In certain preferred embodiments, an inducible expression system is be used to control expression of an exogenous nucleic acid sequence in the transgenic non-human animal. Various inducible systems are known that allow spatial and/or temporal control of expression and that are functional in vivo in transgenic animals. In one embodiment, the inducible system comprises: i) an acceptor sequence that responds to a compound or polypeptide (for example, a TRE or a recombinase site) and regulates expression of a polypeptide of interest from a nucleic acid sequence; and ii) an activator (for example, a transactivator or a recombinase) that acts on the acceptor sequence to induce expression of the desired nucleic acid sequence.

In one embodiment, the acceptor is an inducible expression control sequence comprising an inducible expression element (such as a TRE) and optionally one or more expression control sequences, such as, but not limited to, a promoter. In one embodiment, the acceptor is a TRE and a TRE-associated promoter or a recombinase site. In one embodiment, the activator is a transactivator, such as tTA or rtTA, or a recombinase.

A number of inducible expression control sequences are known in the art and can be used to generate the transgenic non-human animals of the present disclosure. In certain embodiments, the inducible expression control sequence is monocistronic, directing the expression of a polypeptide from nucleic acid sequence (for example, a mutant POLG1 polypeptide). In certain aspects of this embodiment, the inducible expression control sequence comprises a tetracycline response element (TRE). The inducible expression control sequence may comprise additional elements as is known in the art or as described herein. In certain aspects of this embodiment, the inducible expression control sequence comprises a TRE and a TRE-associated promoter. In certain aspects of this embodiment, the inducible expression control sequence comprises a TRE and a constitutive TRE-associated promoter. In certain aspects of this embodiment, the inducible expression control sequence comprises a TRE and a cytomegalovirus (CMV) minimal TRE-associated promoter.

In certain embodiments, the inducible expression control sequence is bicistronic, directing the expression of two polypeptides from two separate nucleic acid sequences (for example, a mutant POLG1 polypeptide and a reporter polypeptide). In certain aspects of this embodiment, the inducible expression control sequence is bicistronic and comprises a TRE to direct the expression of the two polypeptides. The inducible expression control sequence may comprise additional elements as is known in the art or as described herein. In certain aspects of this embodiment, the inducible expression control sequence is bicistronic and comprises a TRE and two TRE-associated promoters flanking the TRE to direct the expression of the two polypeptides. In certain aspects of this embodiment, the inducible expression control sequence comprises a TRE and two TRE-associated promoters, wherein at least one promoter is a constitutive promoter. In certain aspects of this embodiment, the inducible expression element comprises a TRE and two TRE-associated promoters, wherein at least one promoter is CMV minimal promoter.

The inducible expression control sequence directs the expression of a mutant POLG1 polypeptide only under defined conditions (i.e., when the inducible expression control sequence is induced, such as by the presence of a transactivator). For example, when the inducible expression control sequence comprises a TRE, the inducible expression control sequence is induced when a tetracycline-responsive transactivator is present (either in the presence or absence of an inducer compound as described herein).

One example of an inducible expression system is the tetracycline-regulated gene expression system (Tet-system). The Tet-system is a well characterized system that has been validated in a number of animal models and maximized to allow for tight control of transgene expression. As the Tet-system has been used in various animal model, it is possible to obtain various cells and tissues from such animals, including, but not limited to, stem cells, embryonic cells and tissues, and adult cells and tissue, for use in various in vitro and/or in vivo assays.

The Tet-system has advantages over Cre, FRT, and ER (estrogen receptor) conditional gene expression systems. In the Cre and FRT systems, activation or knockout of the gene is irreversible once recombination is accomplished, whereas, in Tet and ER systems, it is reversible. The Tet-system has very tight control on expression, whereas the ER system is somewhat leaky. However, the Tet-system, which depends on transcription and subsequent translation of a target gene, is not as fast-acting as the ER system, which stabilizes the already-expressed target protein upon hormone administration. Also, since the Tet-operator sequence is naturally absent from mammalian cells, pleiotropy is thought to be minimized compared to hormonal methods of control.

The Tet-system utilizes an inducible expression system of prokaryotic origin with which has been adapted for use in mammalian cells. The Tet-system comprises two components: i) a tetracycline response element (TRE) that regulates expression of a desired nucleic acid sequence (the inducible expression element) and optionally an additional expression control sequence such as a promoter; and ii) a Tet-transactivator that is responsive to an inducer compound. The TRE is composed of seven repeats of a specific-binding site (the Tet-operator) for the Tet-transactivator placed immediately upstream of a promoter to drive expression of a gene. The Tet-transactivator binds the Tet-operator either in the presence or absence of an inducer compound (for example, tetracycline or a tetracycline derivative, typically doxycycline).

In one embodiment, the Tet-transactivator generally comprises a mutated Tet repressor, (TetR) domain fused to the VP16 trans-activator protein from herpes simplex virus to create a transcriptional activator which is regulated by tetracycline or a tetracycline derivative, typically doxycycline (the inducer compound). The Tet-system comes in a variety of different forms, the two most commonly used of which are Tet-On and Tet-Off. In the Tet-Off system, the Tet-transactivator (tTA) promotes expression from the TRE and TRE-associated promoter(s) only in the absence of tetracycline or a derivative thereof (most commonly doxycycline). In the Tet-On system, a reverse Tet-transactivator (rtTA) is used that promotes expression from the TRE and TRE-associated promoter(s) only in the presence of tetracycline or a derivative thereof. Newer variants of rtTA (for example, rtTA^{m2}, rtTA^{m3} and rtTA3) provide precise control of expression, even at low concentrations of tetracycline or a derivative thereof. More than 150 mouse strains that express rtTA in a tissue-, cell type-, or stem cell-specific manner are available for use in producing a transgenic non-human animal as described herein (see Dow et al. PLoS One, e95236, 2014). Therefore, the Tet-system allows temporal control of gene expression (such as from a transgene) in vivo depending on the administration of tetracycline or a tetracycline derivative.

A modified Tet-system is the T-REx system (Life Technologies). In the T-Rex system the nucleic acid sequence of interest is flanked by an upstream promoter and two TetO2 sites. Expression of the nucleic acid sequence of interest is repressed by the high affinity binding of homodimers of the Tet-repressor to each TetO2 sequences in the absence of tetracycline or a tetracycline derivative. Introduction of tetracycline or a tetracycline derivative results in binding of one tetracycline/derivative on each Tet-repressor homodimer followed by release of TetO2 by the Tet-repressor homodimers. Unbinding of Tet-repressor homodimers and TetO2 result in expression of the nucleic acid sequence of interest.

The Cre/lox system uses the Cre recombinase, which catalyzes site-specific recombination by crossover between two Cre recognition sequences, the loxP sites in vivo. A nucleic acid sequence introduced between the two loxP sequences (termed a “floxed” sequence) oriented in the same direction is excised by Cre-mediated recombination. Control of Cre expression in a transgenic animal, either constitutively or using either spatial control (with a tissue- or cell-specific promoter) or temporal control (with an inducible system), provides control of excision between the two loxP sites. The Cre/lox system may be used for gene inactivation or protein expression purposes. For example, a floxed inhibitor sequence, for example, a stop codon, may inserted between a promoter sequence and a nucleic acid sequence of interest. Genetically modified non-human animals do not express the nucleic acid sequence of interest until Cre is expressed, leading to excision of the floxed inhibitor sequence. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

In genetics, Flp-FRT recombination is a site-directed recombination technology, increasingly used to manipulate an organism's DNA under controlled conditions in vivo. It is analogous to Cre-lox recombination but involves the recombination of sequences between 34 base pair FRT sites by the recombinase Flp. Control of Flp expression in a transgenic animal, either constitutively or using either spatial control (with a tissue- or cell-specific promoter) or temporal control (with an inducible system), provides control of excision between the two FRT sites. The Flp-FRT system may as described above for the Cre/lox system. Inducible Flp recombinases may also be used as described for the Cre recombinase.

Two or more inducible systems may be used in combination if desired. The tetracycline-inducible system and the Cre/loxP recombinase system (either constitutive or inducible) may be used together for example. A method to use these systems in vivo involves generating two lines of genetically modified animals. One animal line expresses the activator (for example, tTA, rtTA, or Cre recombinase) under the control of a selected promoter. The other animal line expresses the acceptor sequence which controls the expression of the polypeptide of interest (for example, an inducible expression control sequence, such as a TRE and a TRE-associated promoter), that is induced by the activator (for example, a tTA/rtTA or a floxed nucleic acid sequence). Mating the two strains of mice provides control of gene expression.

Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

### Promoters

Promoters useful in driving expression of a transactivator, including a tetracycline responsive transactivator, thereby resulting in expression of a polypeptide of interest (such as a mutant POLG1), include, but are not limited to, constitutive promoters, tissue-specific promoters, development-stage-specific promoters, and inducible promoters. Constitutive promoters direct expression in virtually all tissues and are largely, if not entirely, independent of environmental and developmental factors. For example, a CAG promoter, a CMV promoter, a herpes simplex virus thymidine kinase (HSV-TK) promoter, the SV40 promoter, or 3-phosphoglycerate kinase (PGK) promoter can be used. Tissue-specific or development-stage-specific promoters direct the expression of the transactivator in specific tissue(s) or at certain stages of development. Inducible promoters are not conditioned to endogenous factors but to environmental conditions and external stimuli that can be artificially controlled. Modulating factors can include, for example, a chemical compound, light, oxygen levels, heat, cold and wounding. Since some of these factors are difficult to control outside an experimental setting, promoters that respond to chemical compounds, not found naturally in the organism of interest, are of particular interest. Along those lines, promoters that respond to antibiotics, metals, alcohols, steroids, among other compounds, are useful to allow the induction of the transactivator, and subsequent polypeptide expression following activation of the transactivator, at will and independently of other biotic or abiotic factors. In certain preferred embodiments, the promoter driving the expression of the transactivator is a constitutive promoter. In certain preferred embodiments, the promoter driving the expression of the transactivator is a CMV promoter. In certain preferred embodiments, the promoter driving the expression of the transactivator is a CAG promoter. In certain embodiments, both are used.

Promoters useful in driving expression of a polypeptide of interest (for example, as part of an inducible expression control sequence), for example, a mutant POLG1, include, but are not limited to, constitutive promoters, tissue-specific promoters, development-stage-specific promoters, and inducible promoters as described above. In some embodiments, a constitutive promoter is used. For example, a CAG promoter, a CMV promoter, a HSV-TK promoter, the SV40 promoter, or a PGK promoter can be used. In some embodiments, a promoter with reduced activating activity (i.e., a weak promoter) is used so that expression of the polypeptide of interest is minimal or absent in the absence of induction. In some embodiments, a minimal CMV promoter is used.

In certain embodiments, a tissue specific promoter is used to drive the expression of the transactivator or the polypeptide of interest. A number of tissue specific promoters are known in the art and any may be used as a promoter in the methods described. Using a tissue specific promoter allows the expression of the transactivator or the polypeptide of interest in a desired tissue, organ, or cell for which the promoter is specific. In certain embodiments, the transactivator or the polypeptide of interest is expressed specifically in a bodily system, such as, but not limited to, the cardiovascular, digestive, endocrine, immune, integumentary, lymphatic, muscular, nervous, reproductive, respiratory, skeletal and urinary systems. In certain embodiments, the transactivator or the polypeptide of interest herein is expressed a particular cell type. In certain embodiments, the transactivator or the polypeptide of interest herein is expressed specifically in the skin, a specific layer of the skin (such as, but not limited to, the epidermis, dermis, and hypodermis), a specific layer of the epidermis (such as, but not limited to, the stratum corneum, stratum lucidium, stratum granulosum, stratum spinosum, and stratum basale), the heart, lung, brain, a reproductive tissue, liver, bladder, kidneys, stomach, or intestines In certain embodiments, the tissue specific promoter is a skin promoter. Specific skin-specific promoters include, but are not limited to, K5, K14, and involucrin for the epidermis, tyrosinase for a melanocyte, and α-V integrin for the dermis.

### Non-Human Animal Selection

A number of methods are known in the art for introducing a transgene into a non-human animal. In one exemplary embodiment, the one or more transgenes are introduced into an egg (for example, a one-cell stage egg) of the non-human animal, implanting the egg in a pseudopregnant foster mother, and allowing the egg to develop to thereby produce the non-human, transgenic animal comprising the transgene.

Standard breeding techniques can be used to create animals that are homozygous or heterozygous for the desired transgene(s). Homozygosity may not be required, however. Once transgenic animal have been generated, expression of an exogenous nucleic acid can be assessed using standard techniques or using a reporter polypeptide. Initial screening can be accomplished by Southern blot analysis to determine whether or not integration of the construct has taken place. Polymerase chain reaction (PCR) techniques also can be used in the initial screening.

Expression of a polypeptide of interest from a nucleic acid sequence encoding the polypeptide in the tissues of a transgenic non-human animal can be assessed using techniques that include, for example, Northern blot analysis of tissue samples obtained from the animal, in situ hybridization analysis, Western analysis, immunoassays such as enzyme-linked immunosorbent assays, and RT-PCR. Furthermore, fluorescence monitoring may also be used in the initial screening to determine the expression of a desired polypeptide.

Founder animals (F0 generation) may be produced by cloning and other methods described herein. The founders can be homozygous for a genetic modification. Similarly, founders can also be made that are heterozygous. The founders may be genomically modified, meaning that the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

The transgenic non-human animals of the disclosure can be made, for example, by introducing one or more transgenes into a non-human animal, the one or more transgenes comprising: i) an inducible expression control sequence operably linked to a nucleic acid sequence encoding a mutant POLG1 polypeptide; ii) an inducible expression control sequence (preferably the same as in i)) operably linked to a nucleic acid sequence encoding an additional polypeptide (such as, but not limited to, a reporter polypeptide); and iii) a second expression control sequence operably linked to a nucleic acid sequence encoding a transactivator capable of activating the inducible expression control sequence, leading to expression of the mutant POLG1 polypeptide. Elements i) and ii), i) and iii), or 1) and ii) and iii) may be present on a single transgene or on two or more transgenes.

In at least one embodiment, the transgene comprising a nucleic acid sequence encoding a mutant POLG1 is flanked by attB sites and inserted at a specific locus containing one or more tandem attP sites. In another embodiment, the transgene comprising a nucleic acid sequence encoding a mutant POLG1 is operably linked with an inducible expression control sequence (such as a TRE and a TRE-associated promoter), to conditionally drive expression of the mutant POLG1 utilizing the Tet-Off or Tet-On system, and is optionally flanked by attB sites and inserted at a specific locus containing one or more tandem attP sites. This allows for a transgene to be inserted into a predetermined chromosomal locus and/or also allows for conditional expression of the transgene.

In some embodiments, the transgene(s) is(are) inserted at a specific and/or non-disruptively chromosomal locus, such as, but not limited to, the Hip11, the ChAT, and the ROSA26 loci. A site specific chromosomal locus can be targeted by the transgene utilizing site-specific integrase-mediated transgenesis. Briefly, a single-copy transgene is inserted into a predetermined chromosomal locus with high efficiency (up to about 40%). The method utilizes an integrase, such as, but not limited to, the bacteriophage phiC31 integrase, to catalyze recombination between one or two attB sites in the transgene with one or more tandem attP sites that are previously inserted into a specific locus in the animal's genome via standard homologous recombination-based methods in embryonic stem cells.

In some embodiments, the transgene(s) is(are) inserted at a non-specific and/or non-disruptive chromosomal locus.

### Non-Human Animals

The present disclosure provides genetically modified non-human animals that contain a nucleic acid sequence encoding a mutant POLG1 polypeptide and that express the mutant POLG1 polypeptide in a controlled manner. In one embodiment, the mutant POLG1 polypeptide is expressed ubiquitously (in every cell of the non-human animal). In another embodiment, the mutant POLG1 polypeptide is expressed in a specific tissue or set of tissues or in a specific cell type.

In a first embodiment, the non-human animal contains a single transgene integrated into the genome of the non-human animal. In embodiments where a single transgene is used, the single transgene may comprise: i) an inducible expression control sequence operably linked to a nucleic acid sequence encoding a mutant POLG1 polypeptide and optionally to a nucleic acid sequence encoding an additional polypeptide (such as, but not limited to, a reporter polypeptide); and ii) a second expression control sequence, an excisable inhibitor sequence, and a nucleic acid sequence encoding a transactivator capable of activating the inducible expression element, wherein the nucleic acid sequence encoding the transactivator is operably linked to the second expression control sequence only when the excisable inhibitor sequence is excised. In this manner, the nucleic acid sequence encoding the transactivator is not operably linked to the second expression control sequence in the presence of the excisable inhibitor sequence and the transactivator polypeptide is not expressed. A number of methods may be used to excise the inhibitor sequence. For example, the inhibitor sequence may be flanked on each side by recognition sequences for a recombinase such as, but not limited to, Cre or Flp.

In certain aspects of this embodiment, the transgene is inserted at a specific and/or non-disruptively chromosomal locus, such as, but not limited to, the Hip11, the ChAT, and the ROSA26 loci. In certain aspects of this embodiment, the transgenes is inserted at a non-specific and/or non-disruptively chromosomal locus.

In a specific aspect of this embodiment, the present disclosure provides for a transgenic, non-human animal comprising a transgene integrated into a genome of the animal, the transgene comprising an inducible expression control sequence and a first nucleotide sequence encoding a mutant POLG1 polypeptide, wherein the first nucleotide sequence is operably linked to the inducible expression control sequence.

In one aspect of this embodiment, the inducible expression control sequence comprises a TRE. In another aspect of this embodiment, the inducible expression control sequence comprises a TRE and a TRE-associate promoter operably linked to the TRE. In another aspect of this embodiment, the inducible expression control sequence comprises a TRE and two TRE-associated promoters operably linked to the TRE. As described herein, a number of promoters may be used in the inducible expression control sequence. A suitable promoter is a promoter with reduced activating activity (i.e., a weak promoter). A further suitable promoter is a minimal CMV promoter.

In further aspect of this embodiment, the transgene further comprises a second nucleic acid sequence encoding an additional polypeptide, such as reporter polypeptide (including, but not limited to, a fluorescent reporter polypeptide such as GFP) operably linked to an inducible expression control sequence. The expression of the first and second nucleic acid sequences may be under the control of the same inducible expression control sequence. Alternatively, the expression of the first and second nucleic acid sequences may be under the control of different inducible expression control sequences. When expression of the first and second nucleic acid sequences is under the control of the same inducible expression control sequence, the inducible expression control sequence may be bicistronic such that expression of the first and second nucleic acid sequences is at least partially coordinated and dependent on the same mode of induction.

In further aspect of this embodiment, the transgene further comprises a second expression control sequence, an excisable inhibitor sequence, and a third nucleotide sequence encoding a transactivator capable of activating the inducible expression control sequence, wherein the third nucleic acid sequence is operably linked to the second expression control sequence only when the excisable inhibitor sequence is excised. In this aspect, the inhibitor sequence prevents the second expression control sequence and the third nucleic acid sequence from being operably linked such that the transactivator expressed by the third nucleic acid sequence is not expressed in the presence of the inhibitor sequence. A typical inhibitor sequence may be a stop cassette containing termination codons in all reading frames. The excisable inhibitor sequence is preferably flanked on each side by a recognition sequences for a recombinase allowing excision of the inhibitor sequence. Suitable recognition sites include, but are not limited to, loxP sites recognized by the Cre recombinase and FRT sites recognized by the Flp recombinase.

The transactivator encoded by the third nucleic acid sequence activates the inducible expression control sequence under defined conditions and activates expression of the first nucleic acid sequence. When the inducible expression control sequence comprises a TRE, the transactivator is preferably a tetracycline-responsive transactivator, such as tTA or rtTA. When the expression control element comprises a TRE, the tetracycline-responsive transactivator is preferably rtTA.

In those aspects where the tetracycline-responsive transactivator is tTA, the tTA is expressed from the third nucleic acid sequence upon excision of the excisable inhibitor sequence and activation of the second expression control sequence. The tTA then activates the inducible expression control sequence in the absence of an inducer compound, thereby inducing expression of the mutant POLG1 polypeptide and/or the reporter polypeptide encoded by the first and second nucleic acid sequences, respectively.

In those aspects where the tetracycline-responsive transactivator is rtTA, the rtTA is expressed from the third nucleic acid sequence upon excision of the excisable inhibitor sequence and activation of the second expression control sequence. The rtTA then activates the inducible expression control sequence in the presence of an inducer compound, thereby inducing expression of the mutant POLG1 polypeptide and/or the reporter polypeptide encoded by the first and second nucleic acid sequences, respectively.

The second expression control sequence comprises a transactivator promoter and optionally other sequences as described herein. The transactivator promoter is selected from the group consisting of: a constitutive promoter, a tissue-specific promoter, a development-stage-specific promoter, and an inducible promoter. When ubiquitous expression of the mutant POLG1 polypeptide is desired a constitutive promoter may be used. Suitable constitutive promoters are known in the art and described herein, such as, but not limited to, a CAG promoter, a CMV promoter, a HSV-TK promoter, the SV40 promoter, or 3-phosphoglycerate kinase (PGK) promoter. In certain preferred aspects, the transactivator promoter is a CMV promoter, a CAG promoter, or both.

When tissue-specific expression of the mutant POLG1 polypeptide is desired, the transactivator promoter may be a tissue-specific promoter. Tissue specific promoters are well known in the art and any known tissue specific promoter may be used. In certain aspects, the POLG1 polypeptide is expressed specifically in the skin, a specific layer of the skin (such as, but not limited to, the epidermis, dermis, and hypodermis), a specific layer of the epidermis (such as, but not limited to, the stratum corneum, stratum lucidium, stratum granulosum, stratum spinosum, and stratum basale), the heart, lung, brain, a reproductive tissue, liver, bladder, kidneys, stomach, or intestines In certain aspects, the tissue specific promoter is a skin promoter. Specific skin-specific promoters include, but are not limited to, K5, K14, and involucrin for the epidermis, tyrosinase for a melanocyte, and α-V integrin for the dermis.

In a second embodiment, the non-human animal contains more than 1 transgene, such as two transgenes, integrated into the genome of the non-human animal. In embodiments where two transgenes are used, the first transgene may comprise an inducible expression control sequence operably linked to a nucleic acid sequence encoding a mutant POLG1 polypeptide and optionally a nucleic acid sequence encoding an additional polypeptide (such as, but not limited to, a reporter polypeptide) and the second transgene may comprise a second expression control sequence operably linked to a nucleic acid sequence encoding a transactivator capable of activating the inducible expression control sequence. Non-human animals comprising two transgenes can be created by appropriate mating of a first and second transgenic animal comprising the appropriate transgenes.

In certain aspects of this embodiment, the transgene are inserted at a specific and/or non-disruptively chromosomal locus, such as, but not limited to, the Hip11, the ChAT, and the ROSA26 loci. In certain aspects of this embodiment, the transgenes are inserted at a non-specific and/or non-disruptively chromosomal locus.

In a specific aspect of this embodiment, the present disclosure provides for a transgenic, non-human animal comprising a first transgene integrated into a genome of the animal, the first transgene comprising an inducible expression control sequence and a first nucleotide sequence encoding a mutant POLG1 polypeptide, wherein the first nucleotide sequence is operably linked to the inducible expression control sequence.

In one aspect of this embodiment, the inducible expression control sequence comprises a TRE. In another aspect of this embodiment, the inducible expression control sequence comprises a TRE and a TRE-associate promoter operably linked to the TRE. In another aspect of this embodiment, the inducible expression control sequence comprises a TRE and two TRE-associated promoters operably linked to the TRE. As described herein, a number of promoters may be used in the inducible expression control sequence. A suitable promoter is a promoter with reduced activating activity (i.e., a weak promoter). A further suitable promoter is a minimal CMV promoter.

In further aspect of this embodiment, the first transgene further comprises a second nucleic acid sequence encoding an additional polypeptide, such as reporter polypeptide (including, but not limited to, a fluorescent reporter polypeptide such as GFP) operably linked to an inducible expression control sequence. The expression of the first and second nucleic acid sequences may be under the control of the same inducible expression control sequence. Alternatively, the expression of the first and second nucleic acid sequences may be under the control of different inducible expression control sequences. When expression of the first and second nucleic acid sequences is under the control of the same inducible expression control sequence, the inducible expression control sequence may be bicistronic such that expression of the first and second nucleic acid sequences is at least partially coordinated and dependent on the same mode of induction.

In a specific aspect of the second embodiment, the second transgene comprises a second expression control sequence and a third nucleotide sequence encoding a transactivator capable of activating the inducible expression control sequence and operably linked to the second expression control sequence, wherein the transactivator is expressed upon activation of the second expression control sequence.

The transactivator encoded by the third nucleic acid sequence activates the inducible expression control sequence under defined conditions and activates expression of the first nucleic acid sequence. When the inducible expression control sequence comprises a TRE, the transactivator is preferably a tetracycline-responsive transactivator, such as tTA or rtTA. When the expression control element comprises a TRE, the tetracycline-responsive transactivator is preferably rtTA.

In those aspects where the tetracycline-responsive transactivator is tTA, the tTA is expressed from the third nucleic acid sequence upon excision of the excisable inhibitor sequence and activation of the second expression control sequence. The tTA then activates the inducible expression control sequence in the absence of an inducer compound, thereby inducing expression of the mutant POLG1 polypeptide and/or the reporter polypeptide encoded by the first and second nucleic acid sequences, respectively.

In those aspects where the tetracycline-responsive transactivator is rtTA, the rtTA is expressed from the third nucleic acid sequence upon excision of the excisable inhibitor sequence and activation of the second expression control sequence. The rtTA then activates the inducible expression control sequence in the presence of an inducer compound, thereby inducing expression of the mutant POLG1 polypeptide and/or the reporter polypeptide encoded by the first and second nucleic acid sequences, respectively.

The second expression control sequence comprises a transactivator promoter and optionally other sequences as described herein. The transactivator promoter is selected from the group consisting of: a constitutive promoter, a tissue-specific promoter, a development-stage-specific promoter, and an inducible promoter. When ubiquitous expression of the mutant POLG1 polypeptide is desired a constitutive promoter may be used. Suitable constitutive promoters are known in the art and described herein, such as, but not limited to, a CAG promoter, a CMV promoter, a HSV-TK promoter, the SV40 promoter, or 3-phosphoglycerate kinase (PGK) promoter. In certain preferred aspects, the transactivator promoter is a CMV promoter, a CAG promoter, or both.

When tissue-specific expression of the mutant POLG1 polypeptide is desired, the transactivator promoter may be a tissue-specific promoter. Tissue specific promoters are well known in the art and any known tissue specific promoter may be used. In certain aspects, the POLG1 polypeptide is expressed specifically in the skin, a specific layer of the skin (such as, but not limited to, the epidermis, dermis, and hypodermis), a specific layer of the epidermis (such as, but not limited to, the stratum corneum, stratum lucidium, stratum granulosum, stratum spinosum, and stratum basale), the heart, lung, brain, a reproductive tissue, liver, bladder, kidneys, stomach, or intestines In certain aspects, the tissue specific promoter is a skin promoter. Specific skin-specific promoters include, but are not limited to, K5, K14, and involucrin for the epidermis, tyrosinase for a melanocyte, and α-V integrin for the dermis.

In any of the first or second embodiments, or aspects thereof, the mutant POLG1 polypeptide comprises a single mutation. In any of the first or second embodiments, or aspects thereof, the mutant POLG1 polypeptide comprises more than one mutation. In any of the first or second embodiments, or aspects thereof, the mutant POLG1 polypeptide comprises a dominant negative mutation and no other mutation. In any of the first or second embodiments, or aspects thereof, the mutant POLG1 polypeptide comprises a dominant negative mutation and at least one additional mutation. When the mutant POLG1 polypeptide contains a dominant negative mutation, the dominant negative mutation is preferably the D1135A mutation (amino acid numbering provided using the human POLG1 amino acid sequence; SEQ ID NO: 45) or a D to A mutation at a corresponding aspartic acid residue. A corresponding aspartic acid residue may be identified by comparing the sequence of appropriate POLG1 polypeptide to the sequence surrounding amino acid 1135 of the human sequence or the sequences provided in the definitions section. The mutant POLG1 polypeptide may be from any species, suitably modified to contain a mutation of interest (such as, but not limited to, the D to A mutation at a corresponding aspartic acid residue). Suitably, the mutant POLG1 polypeptide is from a human, modified to contain a mutation of interest, such as, but not limited to, a D to A mutation at a corresponding aspartic acid residue. Suitably, the mutant POLG1 polypeptide is from a non-human primate modified to contain a mutation of interest, such as, but not limited to, a D to A mutation at a corresponding aspartic acid residue. Suitably, the mutant POLG1 polypeptide is from a mouse, modified to contain a mutation of interest, such as, but not limited to, a D to A mutation at a corresponding aspartic acid residue. Suitably, the mutant POLG1 polypeptide is from a rat, modified to contain a mutation of interest, such as, but not limited to, a D to A mutation at a corresponding aspartic acid residue. Suitably, the mutant POLG1 polypeptide is from a Pan species, modified to contain a mutation of interest, such as, but not limited to, a D to A mutation at a corresponding aspartic acid residue (such as P. troglodyte; SEQ ID NO: 46). Suitably, the mutant POLG1 polypeptide is from a Mus species, modified to contain a mutation of interest, such as, but not limited to, a D to A mutation at a corresponding aspartic acid residue (such as M. musculus; SEQ ID NO: 47). Suitably, the mutant POLG1 polypeptide is from a Rattus species, modified to contain a mutation of interest, such as, but not limited to, a D to A mutation at a corresponding aspartic acid residue (such as R. norvegicus; SEQ ID NO: 48). Suitably, the mutant POLG1 polypeptide is from a Xenopus species, modified to contain a mutation of interest, such as, but not limited to, a D to A mutation at a corresponding aspartic acid residue (such as X. laevis; SEQ ID NO: 49). Suitably, the mutant POLG1 polypeptide is from a Saccharomyces species, modified to contain a mutation of interest, such as, but not limited to, a D to A mutation at a corresponding aspartic acid residue (such as S. cerivisiae; SEQ ID NO: 50). Suitably, the mutant POLG1 polypeptide is from a Schizosaccharomyces species, modified to contain a mutation of interest, such as, but not limited to, a D to A mutation at a corresponding aspartic acid residue (such as S. pombe; SEQ ID NO: 51). Suitably, the mutant POLG1 polypeptide is from a Nuerospora species, modified to contain a mutation of interest, such as, but not limited to, a D to A mutation at a corresponding aspartic acid residue (such as N. crassa; SEQ ID NO: 52). Suitably, the mutant POLG1 polypeptide is from a Maylandia species, modified to contain a mutation of interest, such as, but not limited to, a D to A mutation at a corresponding aspartic acid residue (such as M. zebra; SEQ ID NO: 53). Suitably, the mutant POLG1 polypeptide is from a Danio species, modified to contain a mutation of interest, such as, but not limited to, a D to A mutation at a corresponding aspartic acid residue (such as D. rerio; SEQ ID NO: 54).

In any of the first or second embodiments, or aspects thereof, the mutant POLG1 polypeptide has the amino acid sequence of SEQ ID NO: 45 modified to contain a mutation of interest (such as, but not limited to, a D1135A mutation), or a sequence that is at least 75% identical to SEQ ID NO: 45 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 45 modified to contain such mutation of interest.

In any of the first or second embodiments, or aspects thereof, the mutant POLG1 polypeptide has the amino acid sequence of SEQ ID NO: 46 modified to contain a mutation of interest (such as, but not limited to, a D183A mutation), or a sequence that is at least 75% identical to SEQ ID NO: 46 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 46 modified to contain such mutation of interest.

In any of the first or second embodiments, or aspects thereof, the mutant POLG1 polypeptide has the amino acid sequence of SEQ ID NO: 47 modified to contain a mutation of interest (such as, but not limited to, a D1164A mutation), or a sequence that is at least 75% identical to SEQ ID NO: 47 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 47 modified to contain such mutation of interest.

In any of the first or second embodiments, or aspects thereof, the mutant POLG1 polypeptide has the amino acid sequence of SEQ ID NO: 48 modified to contain a mutation of interest (such as, but not limited to, a D1162A mutation), or a sequence that is at least 75% identical to SEQ ID NO: 48 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 48 modified to contain such mutation of interest.

In any of the first or second embodiments, or aspects thereof, the mutant POLG1 polypeptide has the amino acid sequence of SEQ ID NO: 49 modified to contain a mutation of interest (such as, but not limited to, a D1104A mutation), or a sequence that is at least 75% identical to SEQ ID NO: 49 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 49 modified to contain such mutation of interest.

In any of the first or second embodiments, or aspects thereof, the mutant POLG1 polypeptide has the amino acid sequence of SEQ ID NO: 50 modified to contain a mutation of interest (such as, but not limited to, a D892A mutation), or a sequence that is at least 75% identical to SEQ ID NO: 50 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 50 modified to contain such mutation of interest.

In any of the first or second embodiments, or aspects thereof, the mutant POLG1 polypeptide has the amino acid sequence of SEQ ID NO: 51 modified to contain a mutation of interest (such as, but not limited to, a D887A mutation), or a sequence that is at least 75% identical to SEQ ID NO: 51 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 51 modified to contain such mutation of interest.

In any of the first or second embodiments, or aspects thereof, the mutant POLG1 polypeptide has the amino acid sequence of SEQ ID NO: 52 modified to contain a mutation of interest (such as, but not limited to, a D941A mutation), or a sequence that is at least 75% identical to SEQ ID NO: 52 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 52 modified to contain such mutation of interest.

In any of the first or second embodiments, or aspects thereof, the mutant POLG1 polypeptide has the amino acid sequence of SEQ ID NO: 53 modified to contain a mutation of interest (such as, but not limited to, a D1097A mutation), or a sequence that is at least 75% identical to SEQ ID NO: 53 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 53 modified to contain such mutation of interest.

In any of the first or second embodiments, or aspects thereof, the mutant POLG1 polypeptide has the amino acid sequence of SEQ ID NO: 54 modified to contain a mutation of interest (such as, but not limited to, a D1099A mutation), or a sequence that is at least 75% identical to SEQ ID NO: 54 modified to contain such mutation of interest. Preferably, the POLG1 polypeptide has an amino acid sequence that is at least 80% identical, 85% identical, 90% identical, 95% identical, 97% identical, or 99% identical to SEQ ID NO: 54 modified to contain such mutation of interest.

In any of the first or second embodiments, or aspects thereof, the mutant POLG1 polypeptide is from a human, modified to contain a naturally occurring variant and optionally a mutation of interest, such as, but not limited to, a D to A mutation at a corresponding aspartic acid residue. Exemplary natural variants include, but are not limited to, R3P, P18S, Q55QQ, Q55QQQ, R193Q, R227W, R232G, R232H, L244P, T251I, G268A, L304R, Q308H, R309L, W312R, P324S, G380D, G431V, L463F, A467T, N468D, Q497H, S511N, G517V, R546C, R2Q, R574W, R579W, P587L, M603L, R627Q, R27W, P648R, E662K, G737R, W748S, A767D, R807C, R807P, Y831C, G848S, R853W, N864S, Q879H, T885S, A889T, T914P, G923D, H932Y, R943H, R953C, Y955C, A957S, R1047Q, G1051R, G1076V, R1096H, R1096C, S1104C, A1105T, V1106I, H1110Y, H1134R, E1136K, R1142W, E1143G, R1146C, S1176L, D1184N, D1186H, K1191N, and Q1236H.

In any of the first or second embodiments, or aspects thereof, the mutant POLG1 polypeptide has the amino acid sequence of SEQ ID NO: 45 modified to contain a naturally occurring variant and optionally a mutation of interest (such as, but not limited to, a D1135A mutation), or a sequence that is at least 75% identical to SEQ ID NO: 45 containing such a natural variant and optional mutation of interest. Preferably, the POLG1 polypeptide has an amino acid sequence that is at least 80% identical, 85% identical, 90% identical, 95% identical, 97% identical, or 99% identical to SEQ ID NO: 45 containing such a natural variant and optional mutation of interest. Exemplary natural variants include, but are not limited to, R3P, P18S, Q55QQ, Q55QQQ, R193Q, R227W, R232G, R232H, L244P, T251I, G268A, L304R, Q308H, R309L, W312R, P324S, G380D, G431V, L463F, A467T, N468D, Q497H, S511N, G517V, R546C, R2Q, R574W, R579W, P587L, M603L, R627Q, R27W, P648R, E662K, G737R, W748S, A767D, R807C, R807P, Y831C, G848S, R853W, N864S, Q879H, T885S, A889T, T914P, G923D, H932Y, R943H, R953C, Y955C, A957S, R1047Q, G1051R, G1076V, R1096H, R1096C, S1104C, A1105T, V1106I, H1110Y, H1134R, E1136K, R1142W, E1143G, R1146C, S1176L, D1184N, D1186H, K1191N, and Q1236H.

In any of the first or second embodiments, or aspects thereof, expression of the mutant POLG1 polypeptide, results in the transgenic non-human animal exhibiting at least one characteristic selected from the group consisting of: reduced mitochondrial (mt) DNA content, reduced mtDNA copy number, changes in mitochondrial protein expression, reduced expression of mitochondrial oxidative phosphorylation complexes, reduced stability of mitochondrial oxidative phosphorylation complexes, skin wrinkles, hair loss, increased epidermal thickness, epidermal hyperplasia, acanthosis, hyperkeratosis, increased expression of at least one gene selected from the group consisting of: NF-κB, COX-2, INF-β1, CCL5, MMP1, MMP2, MMP9, MMP13, IGF1R, VEGF, and MRPS5, decreased expression of TIMP1 and KLOTHO, increased skin inflammation, and aberrant hair follicles.

In any of the first or second embodiments, or aspects thereof, the inducer compound is tetracycline or a tetracycline derivative. Preferably, the inducer compound is doxycycline. Doxycycline requires a significantly lower concentration for complete activation or inactivation (0.01-1 μg/ml vs. 1-2 μg/ml for tetracycline) and has a longer half-life. Other tetracycline derivatives are known in the art. Suitable tetracycline derivatives are described in Krueger, et al., Biotechniques, 37, 546-548, 2004) or can be identified using the methods described therein. In any of the first or second embodiments, or aspects thereof, the transgenic non-human animal may be any non-human animal in the art. Suitable non-human animals include, but are not limited to, a non-human primate, a mouse, a rat, a cow, a pig, a goat, and a sheep. Suitably, the non-human animal is a mouse.

In any of the first or second embodiments, or aspects thereof, the inducer compound is added at a predetermined concentration selected to provide for the expression of the mutant POLG1 polypeptide at a desired level. For example, as the concentration of the inducer compound is increased, the expression of the mutant POLG1 polypeptide increases. Conversely, a decrease in the concentration of the inducer compound can be expected to lead to a decrease in expression of the mutant POLG1 polypeptide. The concentration of the inducer compound can be used to increase or lessen an effect mediated by expression of the mutant POLG1 polypeptide. As such, the concentration of inducer compound can be used to vary the severity of an effect mediated by the mutant POLG1 polypeptide. As used in this discussion, the concentration of inducer compound means the concentration of the inducer compound added to the feed and/or drinking water of the transgenic non-human animal.

In certain embodiments, the concentration of the inducer compound is selected to provide for a fatality rate in the transgenic non-human animal of less than or equal to 50%, less than or equal to 40%, less than or equal to 30, less than or equal to 20%, less than or equal to 10%, or less than or equal to 5%, after 2 months of expression of the mutant POLG1 polypeptide.

In one embodiment, the concentration of the inducer compound is from 200 mg/kg to 300 mg/kg in the feed and from 0 mg/ml to 25 mg/ml in the drinking water. In one embodiment, the concentration of the inducer compound is from 150 mg/kg to 250 mg/kg in the feed and from 0 mg/ml to 25 mg/ml in the drinking water. In one embodiment, the concentration of the inducer compound is from 100 mg/kg to 200 mg/kg in the feed and optionally from 0 mg/ml to 25 mg/ml in the drinking water. In one embodiment, the concentration of the inducer compound is from 75 mg/kg to 150 mg/kg in the feed and optionally from 0 mg/ml to 25 mg/ml in the drinking water.

In one embodiment, the concentration of the inducer compound is from 50 mg/kg to 100 mg/kg in the feed and optionally from 0 mg/ml to 25 mg/ml in the drinking water. In one embodiment, the concentration of the inducer compound is from 25 mg/kg to 50 mg/kg in the feed and from 0 mg/ml to 25 mg/ml in the drinking water.

In one embodiment, the concentration of the inducer compound is from 0 mg/kg to 200 mg/kg in the feed and from 1 mg/ml to 25 mg/ml in the drinking water. In one embodiment, the concentration of the inducer compound is from 0 mg/kg to 200 mg/kg in the feed and from 2 mg/ml to 20 mg/ml in the drinking water. In one embodiment, the concentration of the inducer compound is from 0 mg/kg to 200 mg/kg in the feed and optionally from 4 mg/ml to 15 mg/ml in the drinking water. In one embodiment, the concentration of the inducer compound is from 0 mg/kg to 200 mg/kg in the feed and optionally from 6 mg/ml to 12 mg/ml in the drinking water. In one embodiment, the concentration of the inducer compound is from 0 mg/kg to 200 mg/kg in the feed and optionally from 4 mg/ml to 10 mg/ml in the drinking water. In one embodiment, the concentration of the inducer compound is from 0 mg/kg to 200 mg/kg in the feed and from 2 mg/ml to 8 mg/ml in the drinking water.

### Methods of Screening

The present disclosure further provides for methods of screening to identify and/or develop compounds for the treatment of various diseases and conditions. In one embodiment, the present disclosure provides for identification of agents for the treatment of a disease or condition due, at least in part, to mitochodrial dysfunction, changes in mtDNA copy number and/or concentration, and/or dysfunctional mitochondrial OXPHOS. Such diseases and conditions include, but are not limited to, mtDNA depletion syndromes, skin wrinkles, hair loss, increased epidermal thickness, epidermal hyperplasia, acanthosis, hyperkeratosis, cardiovascular disease, diabetes, cancer, aging, and aging-associated chronic diseases or pathologies, such as, but not limited to age-associated neurological disorders.

In one embodiment, such a method of screening comprises the steps of: a) providing a transgenic non-human animal capable of inducible expression of a mutant POLG1 polypeptide; b) stimulating the expression of the mutant POLG1 polypeptide, wherein stimulating expression of the mutant POLG1 polypeptide induces a desired pathology; c) administering an agent to the transgenic non-human animal either before step b) or after step b); d) determining the effect of the agent on pathology; and e) comparing the effect of the agent to a control animal, wherein a reduction or an increase (as appropriate) in the pathology in the transgenic non-human animal after administration of the agent indicates the agent is a therapeutic agent for the treatment of the pathology.

In another embodiment, the present disclosure provides a method for identifying a therapeutic agent for the treatment of skin wrinkles, the method comprising the steps of: a) providing a transgenic non-human animal capable of inducible expression of a mutant POLG1 polypeptide; b) stimulating the expression of the mutant POLG1 polypeptide, wherein stimulating expression of the mutant POLG1 polypeptide induces skin wrinkles; c) administering an agent to the transgenic non-human animal either before step b) or after step b); d) determining the effect of the agent on skin wrinkles or a parameter of skin wrinkles; and e) comparing the effect of the agent to a control animal, wherein a reduction in skin wrinkles or the parameter of skin wrinkles in the transgenic non-human animal after administration of the agent indicates the agent is a therapeutic agent for the treatment of skin wrinkles.

The parameter of a skin wrinkle may be wrinkle length, wrinkle depth, the number of wrinkles in a defined area, and/or the spacing between wrinkles. The number of wrinkles in a defined area can be determined empirically. In a preferred embodiment, the method for measuring a parameter of skin wrinkles is described in WO2013112974A1. Such a method comprises determining the length of a wrinkle and at least one other measured physical characteristic of a skin wrinkle, such as wrinkle depth, the number of wrinkles in a defined area, and/or the spacing between wrinkles. Further, the skin wrinkles parameters may be used to define a severity level of the skin wrinkle, the severity level being based on a combination of wrinkle length and the at least on other measured physical characteristic of a skin wrinkle.

In another embodiment, the present disclosure provides a method for identifying a therapeutic agent for the treatment of skin wrinkles, the method comprising the steps of: a) providing a transgenic non-human animal capable of inducible expression of a mutant POLG1 polypeptide; b) stimulating the expression of the mutant POLG1 polypeptide, wherein stimulating expression of the mutant POLG1 polypeptide induces skin wrinkles; c) administering an agent to the transgenic non-human animal either before step b) or after step b); d) determining the effect of the agent on the number of skin wrinkles; and e) comparing the effect of the agent to a control animal, wherein a reduction in the number of skin wrinkles in the transgenic non-human animal after administration of the agent indicates the agent is a therapeutic agent for the treatment of skin wrinkles.

The number of wrinkles can be determined empirically. For example, the number of skin wrinkles in a defined area may be used.

In a further embodiment, the present disclosure provides a method for identifying a therapeutic agent for the treatment of hair loss, the method comprising the steps of: a) providing a transgenic non-human animal capable of inducible expression of a mutant POLG1 polypeptide; b) stimulating the expression of the mutant POLG1 polypeptide, wherein stimulating expression of the mutant POLG1 polypeptide induces hair loss; c) administering an agent to the transgenic non-human animal either before step b) or after step b); d) determining the effect of the agent on hair loss; and e) comparing the effect of the agent to a control animal, wherein a reduction in hair loss or an increase in hair growth in the transgenic non-human animal after administration of the agent indicates the agent is a therapeutic agent for the treatment of hair loss.

In a preferred embodiment, hair loss is quantified visually through the use of photographs. The photographs may be taken with the aid of a stereotactic positioning device on which camera is mounted, to assure that the view, magnification and lighting are consistent over different measurement periods.

In any of the described methods of screening, agents can include, but are not limited to, chemical compounds, pharmaceutical compositions, biological compounds and compositions (e.g., proteins, DNA, RNA, siRNAs, vaccines and the like), and microorganisms. Further, the agent may be selected from a library, including a library of agents approved by a regulatory authority such as the FDA.

In any of the described methods of screening, any of the transgenic transgenic non-human animals of the present disclosure may be used.

In any of the described methods of screening, step b) may be accomplished by providing an inducer compound to the transgenic non-human animal or withholding the inducer compound from the transgenic non-human animal.

In any of the described methods of screening, the agent is added before step b). In any of the described methods of screening, the agent is added after step b).

In any of the described methods of screening, the inducer compound is tetracycline or a tetracycline derivative. In any of the described methods of screening, the mutant POLG1 polypeptide may be any mutant POLG1 polypeptide described herein. In certain aspects, the mutant POLG1 polypeptide comprises a dominant negative mutation. In certain aspects, the mutant POLG1 polypeptide comprises a D1135A mutation.

### EXAMPLES

### Example 1—Development of mtDNA-Depleter Mouse

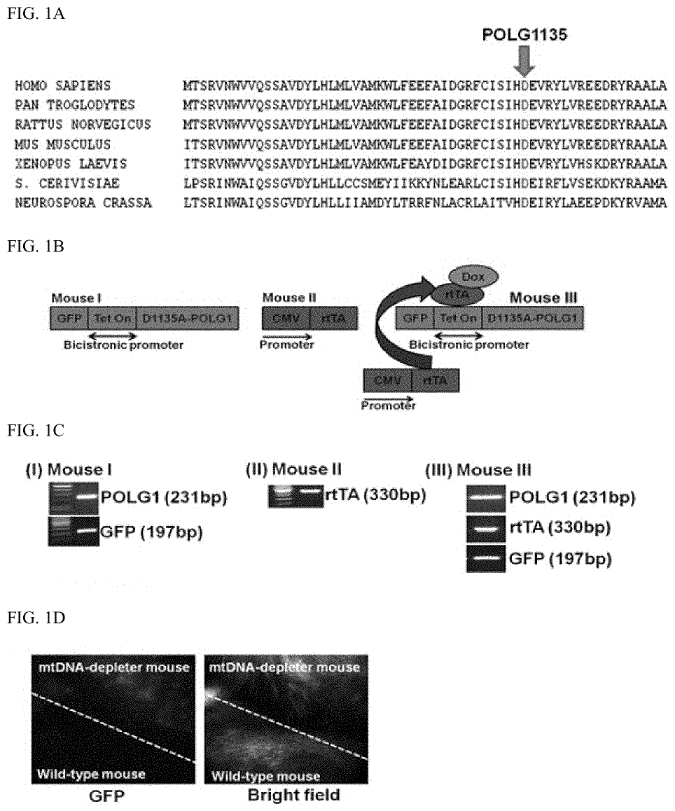

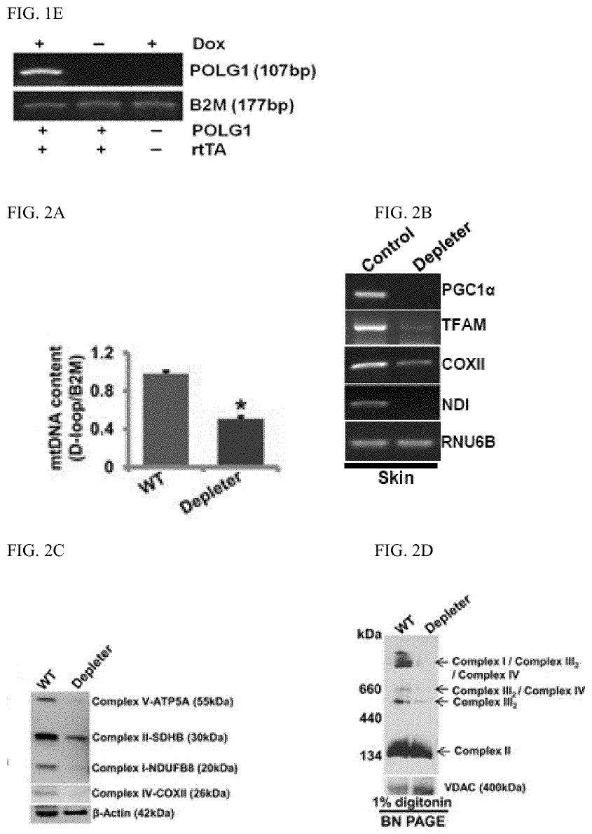

An aspartic acid to alanine amino acid change at the evolutionarily conserved site in the polymerase domain of POLG1 at position 1135 (D1135A-POLG1; POLG1-DN) (FIG.) acts as a DN mutation, and its expression leads to decrease in mtDNA content and mitochondrial activity (Jazayeri et al., J Biol Chem, 278, 9823-9830, 2003; Singh et al., Hum Genet, 54, 516-524, 2009). A Tet-inducible POLG1-DN mouse model was developed with a ubiquitously expressed bidirectional promoter to control the expression of both POLG1-DN and green fluorescence protein (GFP) (Singh et al., Hum Genet, 54, 516-524, 2009). A POLG1-DN-expressing mouse was created by microinjection of the pTRE-Tight-BI-AcGFP1-D1135A-POLG1 construct into the one-cell stage egg from a C57BL/6 mouse. The POLG1-DN-positive founder male mouse (Mouse I) was bred with a female mouse containing the chicken β-actin-reverse tetracycline-controlled transactivator 3 (CAG-rtTA3) female mouse (Mouse II, Jackson Laboratories) to obtain the inducible POLG1-DN transgenic animal (Mouse III) (FIG. 1B). The presence of the DN POLG1, rtTA3, and GFP were verified by polymerase chain reaction (PCR) genotyping (FIG. 1C). The rtTA3 was under the control of the ubiquitously expressed CMV early enhancer element and CAG promoter. The POLG1-DN transgene was turned on by adding doxycycline (dox) in the food (200 mg/kg) and/or drinking water (2 mg/ml in 5% sucrose water) when the mice were 8 weeks of age. The expression of GFP in POLG1-DN transgenic (mtDNA-depleter) animals was also verified by whole-body imaging for GFP after dox-mediated induction (FIG. 1D). The specificity of dox induction was verified by reverse transcription-PCR (RT-PCR) for the expression of POLG1 in the presence and absence of dox (FIG. 1E).

### Example 2—Reduced mtDNA, OXPHOS Super Complexes, and Enzymatic Activities in mtDNA-Depleter Mice

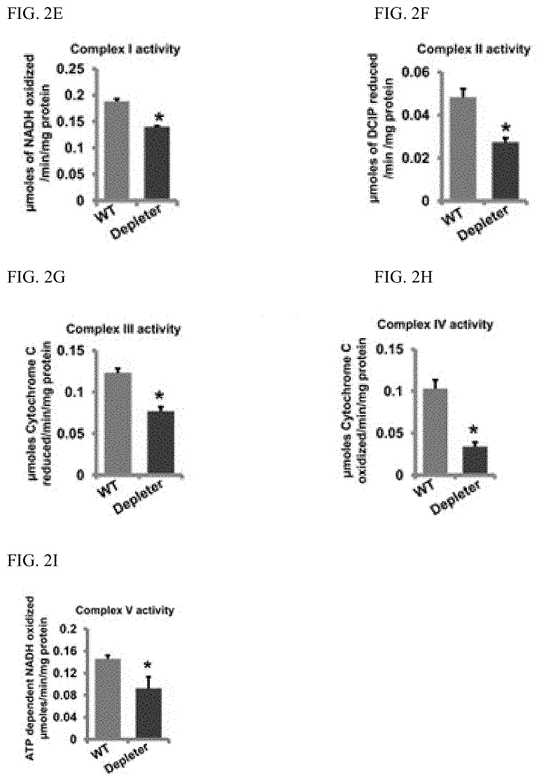

To further characterize the mtDNA-depleter mice, the mtDNA content in different tissues such as the skin (FIG. 2A) and heart, lung, brain, and liver (FIG. 2J) of mtDNA-depleter mice was examined. A significant decrease in mtDNA content in these tissues confirmed the ubiquitous decrease of mtDNA content in mtDNA-depleter mice. mRNA expression of mtDNA-encoded genes and nuclear genome-encoded regulators of mitochondrial biogenesis (PGC1α) and mitochondrial transcription and genome regulation (TFAM), (FIG. 2B), expression of OXPHOS proteins (FIG. 2C), and stability of OXPHOS super complexes (FIG. 2D) were severely reduced in the skin of mtDNA-depleter mice as compared to wild-type littermates. The enzymatic activities of OXPHOS complexes of mitochondria of the skin of mtDNA-depleter mice was also examined. A significant decrease in enzymatic activities of OXPHOS complexes I to V further confirmed mitochondrial dysfunction in mtDNA-depleter mice (FIG. 2E-I). These observations show that ubiquitous expression of D1135A-POLG1 leads to reduced mtDNA content, reduced expression of mitochondrial genes, reduced OXPHOS super complexes stability, and reduced enzymatic activities of OXPHOS complexes in mtDNA-depleter mice.

### Example 3—mtDNA-Depleter Mice Show Inflamed Wrinkled Skin with the Hyperplastic and Hyperkeratotic Epidermis and Alopecia Secondary to Defective Hair Loss

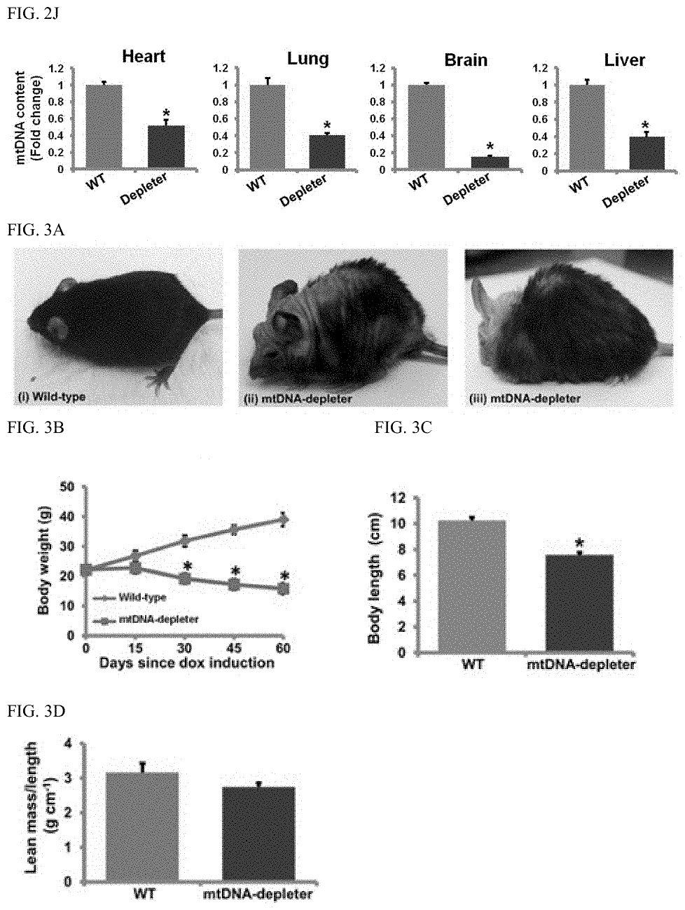

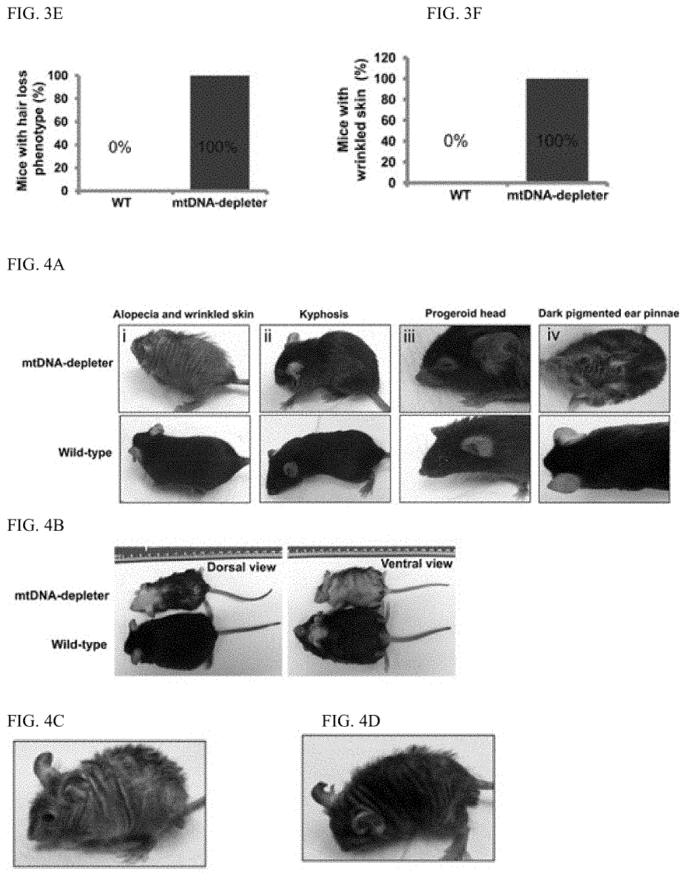

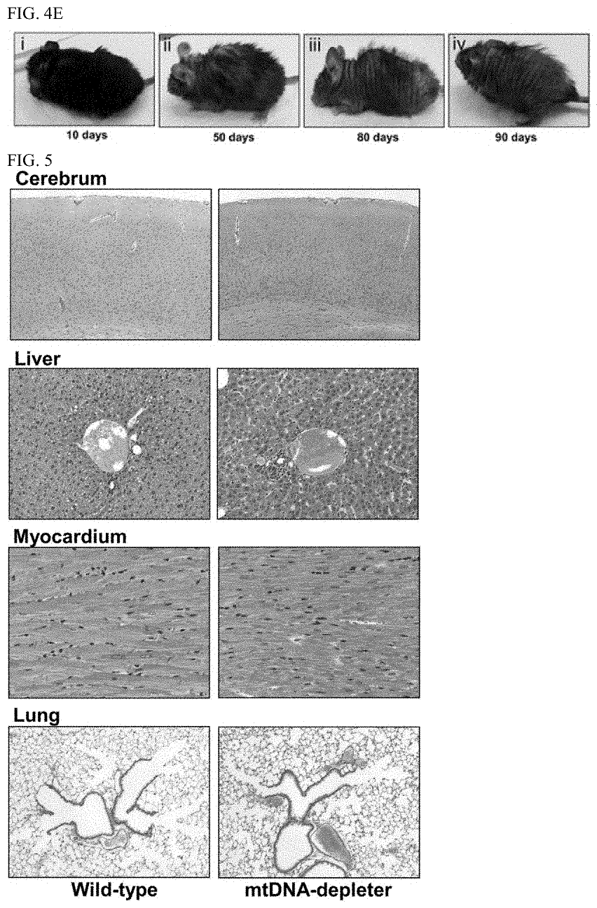

The mtDNA-depleter mice showed a normal appearance until the dox was administered at the age of 8 weeks, resulting in expression of POLG1-DN. After 2 weeks of dox induction, a change in scurf was the first phenotypic symptom. After two more weeks of dox induction gray hair, reduced hair density, hair loss (alopecia), kyphosis, and progeroid head (FIGS. 3 and 4) that were absent in age-matched wild-type littermates. Slowed movements and lethargy were the next line of phenotypic changes observed. These phenotypic changes are reminiscent of phenotypic changes naturally occurring during aging (37-38). The decrease in size and weight of mtDNA-depleter mice was noticeable at this stage (FIGS. 3B, 3C and 4B). No significant change in lean mass to length ratio was observed between wild-type and mtDNA-depleter mice (FIG. 3D). Continuous induction of POLG1-DN transgene led to the death of some of these mice due to severe mitochondrial malfunction. Fifty percent of the total mtDNA-depleter mice examined in this experiment (n=30) died around 40 days of dox induction, while the remaining mtDNA-depleter mice died within 150 days since initiation of dox induction. The modulation of POLG1-DN expression by altering the concentration of the inducer (dox) was observed to reduce lethality of POLG1-DN expression in this animal model.

All the mtDNA-depleter mice that survived at least 30 days of dox induction/POLG1-DN expression showed the development of alopecia (FIG. 3E) and skin wrinkles (FIG. 3F). Further extending the duration of dox induction/POLG1-DN expression resulted in a gradual change in the pattern of hair loss in mtDNA-depleter mice (FIG. 4E). interestingly, the pattern of hair loss was different in male and female mtDNA-depleter mice. While male mice showed dispersed hair loss (FIG. 4C), females represented time-dependent hair loss patterns and overall more severe hair loss compared to male mice (FIG. 4D, 4E). Sex hormones regulate mitochondrial functions and may be an underlying mechanism for gender-specific differences observed in hair loss pattern in mtDNA-depleter mice.

Besides hair loss, skin wrinkles were also evident in all mtDNA-depleter mice (FIGS. 3A and 3F). Female mice exhibited more severe skin wrinkles (FIG. 4D) compared to age-matched male mtDNA-depleter mice (FIG. 4C). No phenotypic changes in the wild-type control group fed on dox diet were noted (FIG. 3A), nor in mtDNA-depleter mice without dox diet (normal diet).

A histopathological evaluation of different tissues of mtDNA-depleter mice was conducted. Interestingly, no significant histological changes were observed in the brain, liver, myocardium, and lung sections of mtDNA-depleter mice after 2 months of dox induction except for a reduction in cell sizes (FIG. 5). Optimal mitochondrial functions are required to maintain the cell size (48). Thus, the reduced cell size is indicative of mitochondrial dysfunction in these organs. At both phenotypic and histological levels, the skin was the first and most affected organ in mtDNA-depleter mice after induction of POLG1-DN.

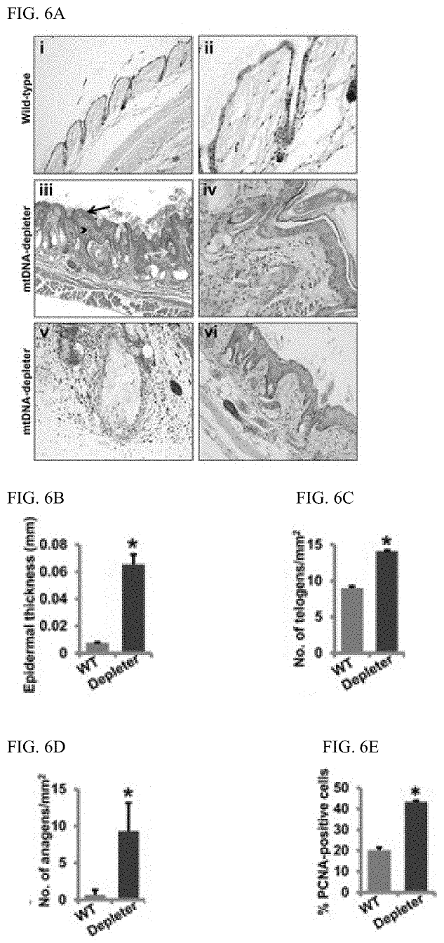

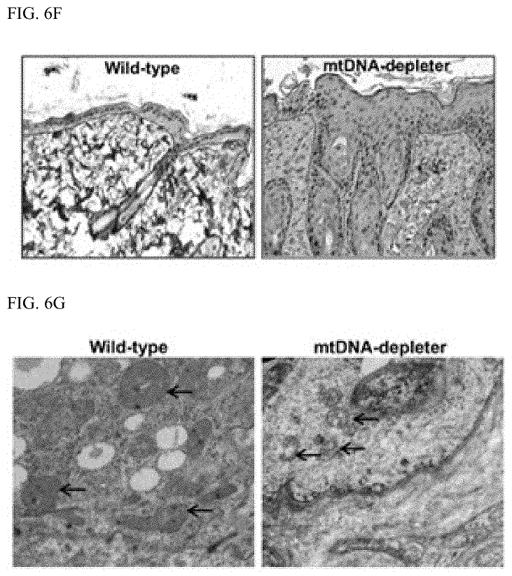

The examination of hematoxylin- and eosin-stained sections of the skin from the wild-type and mtDNA-depleter mice showed striking histological differences in all skin compartments (FIG. 6). The skin from wild-type animals showed typical morphology of telogen skin in which epidermis was thin, composed of 1-2 layers of keratinocytes, the dermis was free of inflammatory infiltrate, and the vast majority of hair follicles were at telogen stage (FIG. 6A, panels i and ii). In striking contrast, the skin from mtDNA-depleter mice after 2 months of dox induction had hyperplastic and hyperkeratotic epidermis, with 4-6 layers of keratinocytes being reminiscent of pathological human epidermis composed of stratum basale, stratum spinosum, and stratum granulosum covered by parakeratotic (predominantly) and compact orthokeratotic scale (FIG. 6A, panels iii-vi). This epidermal hyperplasia is further confirmed by increased expression of proliferation marker PCNA (proliferating cell nuclear antigen) in the skin of mtDNA-depleter mice (FIGS. 6E and 6F). Epidermal hyperplasia is one of the common characteristics of extrinsic aging and is associated with wrinkle formation (51-53). The increased thickness of the epidermis was primarily due to acanthosis and increased the thickness of the stratum spinosum and stratum granulosum, normally not present in mice (FIG. 6B). A considerable hyperkeratosis, including both parakeratosis and orthokeratosis was evident (FIG. 6A, panels iii-vi). The keratinocytic hyperplasia with hyperkeratosis extended into the infundibula of the hair follicles, of which infundibula were occluded by keratotic plugs. This was also associated with formation of follicular cysts, infundibular (epidermoid) type, with some of them ruptured with secondary granulomatous and suppurative inflammation (FIG. 6A, panels iii and v). The majority of the hair follicles showed pathological alterations (FIG. 6A-D). Although there was evidence of follicular cycling and increased number of follicles in both telogen (FIG. 6C) and anagen (FIG. 6D) in mtDNA-depleter mice after 2 months of dox induction compared with wild-type mice, these follicles were aberrant and did not produce normal hair shafts in mtDNA-depleter mice. Instead, follicles contained predominantly keratinaceous debris with only a few developing hair shafts which were fragmented and malformed. Thus, alopecia was not due to loss of hair follicles or cessation of cycling; rather, the follicles were dysfunctional and could not produce a normal hair shaft. Furthermore, abnormal formation of hypertrophic sebaceous glands was noted (FIG. 6A, panels iii and vi) with some areas reminiscent of nevus sebaceous in the human skin.

To establish a link between the changes in the skin and the mtDNA stress, skin samples were analyzed by electron microscopy. Electron microscopic analyses revealed the presence of severely degenerated mitochondria with loss of cristae in the skin of mtDNA-depleter mice after 2 months of dox induction (FIG. 6G). Together, these studies indicate that mtDNA depletion in the whole animal predominantly induces skin wrinkles due to epidermal hyperplasia and hyperkeratosis, and alopecia because of abnormal hair follicle development and the loss of ability to produce hair shafts.

### Example 4—Skin Inflammation in mtDNA-Depleter Mice

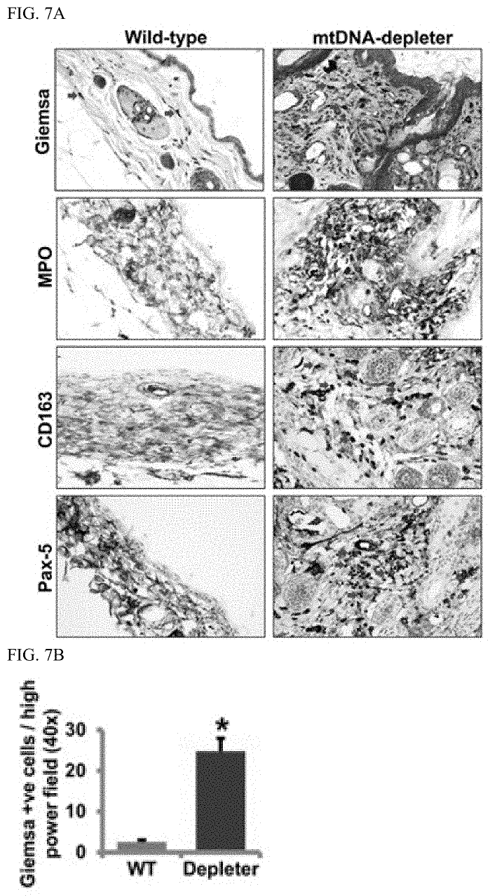

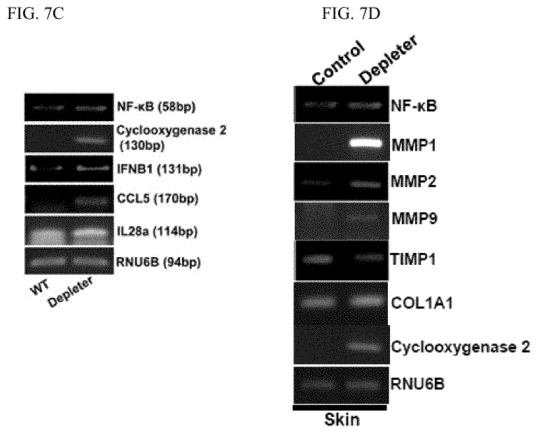

Skin wrinkles are a hallmark of both intrinsic and extrinsic aging of the skin. Alterations in the mitochondrial genome have been associated with the extrinsic aging of the skin (54). The presence of coarse skin wrinkles with marked acanthosis and inflammatory cells in the dermis of mtDNA-depleter mice after 2 months of dox induction presented characteristics akin to the extrinsic aging of skin in human (55). Skin sections were examined for the presence of inflammatory infiltrate in the skin of mtDNA-depleter mice (FIG. 6A). While control mice showed lack of skin inflammation, the mtDNA-depleter mice showed marked mixed dermal inflammatory infiltrate which were also present to a different degree in epidermal and adnexal structures. The infiltrate was predominantly lymphohistiocytic and contained neutrophils, mast cells, and to some degree eosinophils (FIG. 6A). In the areas where follicular cysts were ruptured, neutrophilic infiltrate accompanied by the granulomatous reaction was predominant. To better define the nature of inflammatory cells, immunocytochemistry and histochemistry were performed. These results confirmed presence of increased number of inflammatory cells including mast cells (Giemsa stain-positive cells, FIGS. 7A and 7B), granulocytes (MPO-positive cells, FIG. 7A), macrophages and histiocytes (CD163-positive cells, FIG. 7A), B lymphocytes (Pax-5-positive cells, FIG. 7A), and T lymphocytes (CD3-positive cells, data not shown) in the dermis, as well as in perifollicular and periepidermal location of mtDNA-depleter mice. The skin sections of wild-type mice were predominantly negative for MPO, CD3, CD163, and Pax-5 staining and showed only occasional mast cells. Florid skin inflammatory responses further support the causative link between mitochondrial dysfunction and inflammation. Increased expression of inflammatory genes such as IFNB1, IL28a, and CCL5 in the skin samples of mtDNA-depleter mice after 2 months of dox induction were observed as compared to the skin samples of wild-type mice (FIG. 7C). In addition, increased expression of NF-κB and Cyclooxygenase 2, a NF-κB-regulated mediator of inflammation, in the skin of mtDNA-depleter mice compared to the skin from wild-type littermates (FIG. 7C). These observations indicate that inflammation contributes to the skin aging in mtDNA-depleter mice.

### Example 5—Altered Expression of Matrix Metalloproteinases in the Skin of mtDNA-Depleter Mice

Skin wrinkling is associated with a loss of collagen fibers. A tight balance between the MMPs and their tissue-specific inhibitor TIMP1 is essential to maintain collagen fiber content in the skin. The present disclosure revealed increased expression of MMP2, MMP2, and MMP9 and decreased expression of TIMP1 in mtDNA-depleter mice after 2 months of dox induction (FIG. 7D). Expression of collagen type 1 alpha-1 (COL1A1), an important gene in the de novo synthesis of collagen of the skin, remained unaltered (FIG. 7D). These studies indicate that skin wrinkling in mtDNA-depleter mice occurs as the result of an imbalance between collagen proteolytic enzymes and their inhibitors rather than as a result of a decrease in collagen production.

### Example 6—Altered Expression of Markers of Aging in mtDNA-Depleter Mice

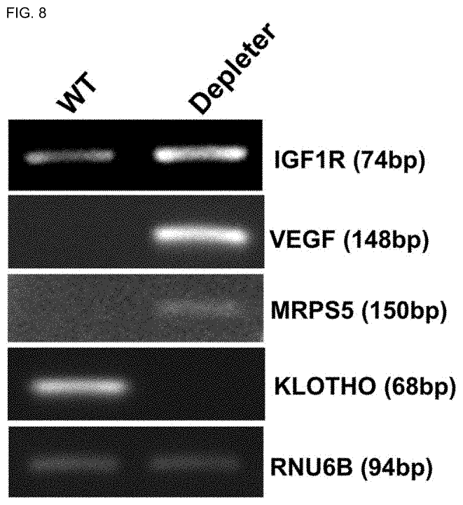

To characterize the association of skin wrinkles and aging at the molecular level, the expression of markers related to intrinsic aging in the skin of mtDNA-depleter mice was examined after 2 months of dox induction. Increased expression of molecular markers of intrinsic aging le IGF1R, VEGF, MRPS5 and decreased expression of Klotho was observed in mtDNA-depleter mice (FIG. 8). These observations suggest that mitochondrial dysfunction induces skin aging through an intrinsic mechanism as well.

### Example 7—Reversal of Wrinkled Skin and Loss of Hair by Repletion of mtDNA

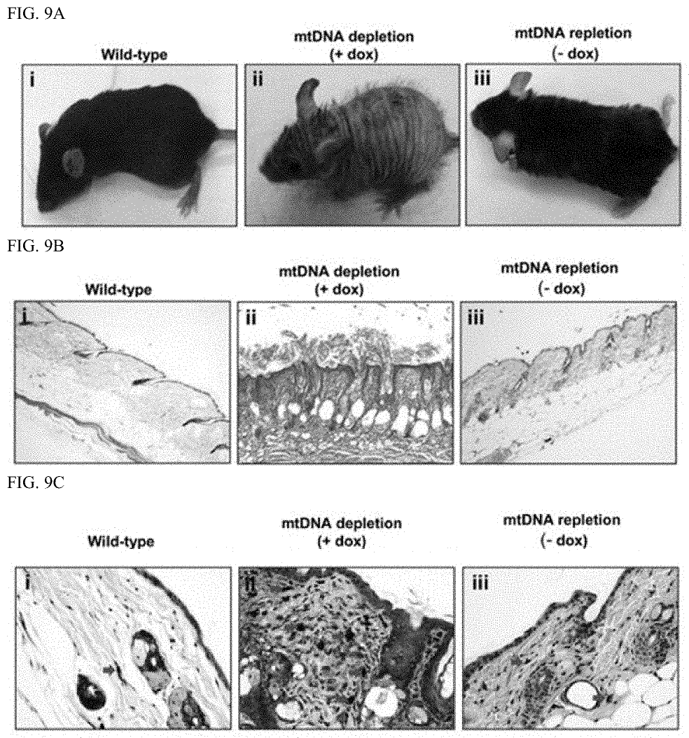

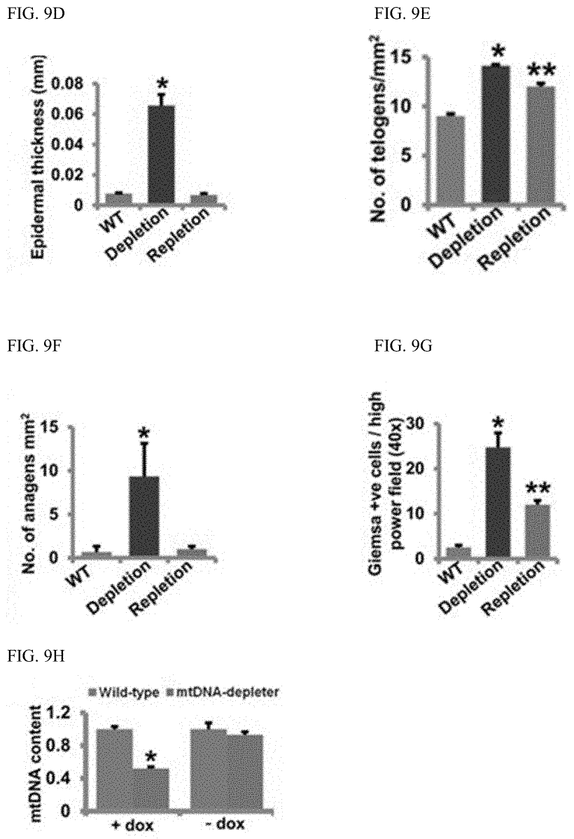

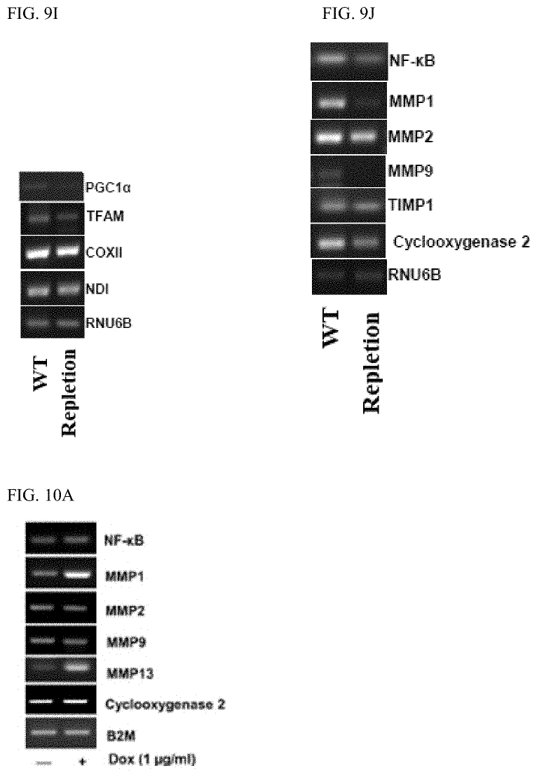

To substantiate that the disruption of mitochondrial function was the underlying cause of the observed phenotypic, histopathologic and molecular changes in the skin of mtDNA-depleter mice, a rescue experiment was conducted. mtDNA content was restored to near normal or normal levels in mtDNA-depleter mice by dox withdrawal (producing a mtDNA-repleter animal). After exposure to dox for two months, there was an induction of typical skin wrinkles and hair loss (FIG. 9A, panel ii). One month after dox withdrawal, the skin wrinkle and hair loss phenotypes reverted, and the mtDNA-repleter animals appeared normal when compared to the age-matched wild-type controls (FIG. 9A, panels i and iii)). The histopathological analysis of skin from the mtDNA-repleter animals showed an almost complete restoration of normalcy in the skin (FIG. 9B). The abnormal sebaceous glands, epidermal hyperplasia (FIG. 9D), follicular dysplasia, and inflammation were absent, but some abnormal hair shafts remained. The number of anagen hair follicles reverted to the wild-type levels (FIG. 9F), and the number of hair follicles in telogen also decreased in the mtDNA-repleter mice compared with mtDNA-depleter mice (FIG. 9E). A significant decrease in the inflammatory infiltrate present in the skin of mtDNA-repleter animals was also observed (FIGS. 9B, 9C, and 9G). The macrophages, granulocytes, and B lymphocyte and T lymphocyte that were present in the skin of mtDNA-depleter mice (FIG. 7A) were predominantly absent in the skin of the mtDNA-repleter mice (data not shown). An increase in mtDNA content to wild-type levels (FIG. 9H) and increased expression of mtDNA-encoded genes (FIGS. 2D and 9I) was also observed in mtDNA-repleter animals. Expression of genes involved in the skin inflammation and wrinkling also reverted to the wild-type levels in mtDNA-repleter animals (FIGS. 7C-D and 9J). These observations demonstrate that mitochondrial dysfunction-induced phenotypical, histopathological, and molecular changes can be reversed by restoration of mitochondrial function and confirm the disruption of mitochondrial function was the underlying cause of the observed phenotypic, histopathologic and molecular changes in the skin of mtDNA-depleter mice.

### Example 8—Reversal of Wrinkled Skin and Loss of Hair by Pharmacological Intervention

Cells from the mtDNA-depleter mice were isolated and cultured in vitro.

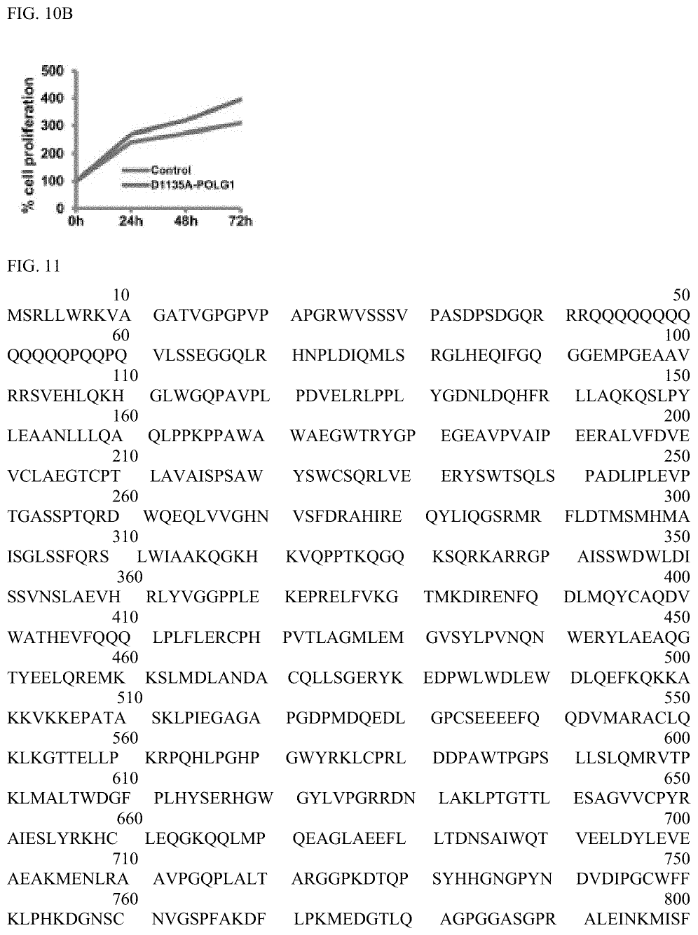

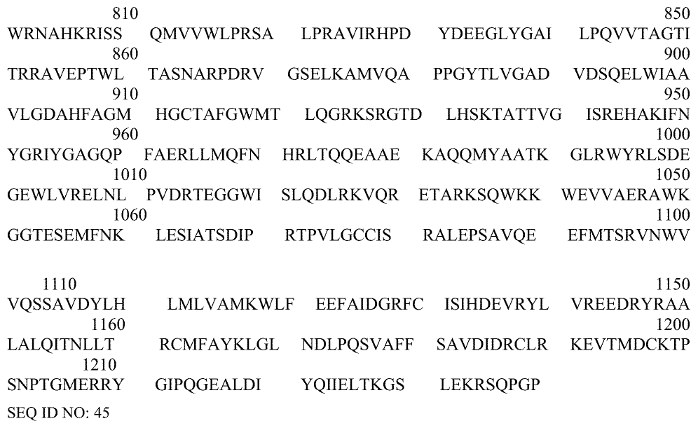

Results from in vitro studies using skin fibroblast cells generated from mtDNA-depleter mouse corroborated the results from the animal studies. Observations of decreased NF-κB expression along with its downstream targets (FIG. 10A) and increased cell proliferation after dox induction (FIG. 10B) mirror the results obtained in whole animal experiments. These data show that various cells and tissue from the mtDNA-depleter mice can be obtained and used for in vitro studies to supplement the data obtained from in vivo studies.

### Materials and Methods

Creation of mtDNA-Depleter Mice

D1135A-POLG1 site-directed mutation was created in the full-length human POLG1 complementary DNA (cDNA) using the site-directed mutagenesis kit (Agilent, Santa Clara, Calif., USA). The primer sequences used for site-directed mutagenesis are as follows, with the mutated site in upper case: D1135A_F:5′-gcatcagcatccatgCGgaggttcgctacctgg-3′ (SEQ ID NO: 1) and D1135A_R:5′-ccaggtagcgaacctcCGcatggatgctgatgc-3′ (SEQ ID NO: 2). Mutations were confirmed by sequencing. D1135A-POLG1 cDNA was subcloned into the dox-inducible mammalian expression vector, pTRE-Tight-BI-AcGFP1 (Clontech, Palo Alto, Calif., USA). To obtain germline transmission of human D1135A-POLG1 (POLG1-DN), microinjection of the pTRE-Tight-BI-AcGFP1-D1135A-POLG1 construct into fertilized oocytes from C57BL/6 mouse was carried out. Potential founders were identified by screening genomic DNA from tail biopsies for the presence of the human Polg1 transgene using the PCR. The heterozygous human POLG1-positive (+/POLG1-DN⁺) founder male mice were mated with CAG-rtTA3 (rtTA) C57BL/6 female mice (Jackson Laboratories, stock no. 016532) to obtain +/POLG1-DN⁺ rtTA⁺ heterozygous transgenic mice. The +/POLG1-DN⁺ rtTA⁺ heterozygous mice were intercrossed to generate homozygous POLG1-DN⁺ rtTA⁺/POLG1-DN⁺ rtTA⁺ mice (mtDNA-depleter mice). This cross resulted in normal litter size (6-7 pups) and Mendelian distributions of genotypes, that is, 1:2:1 distribution of wild-type, heterozygous +/POLG1-DN⁺ or +/rtTA⁺ and homozygous POLG1-DN⁺ rtTA⁺/POLG1-DN⁺ rtTA⁺ showing that homozygosity for POLG1-DN allele does not result in embryonic or postnatal lethality. All the mice were given dox in diet (200 mg/kg diet) and water (2 mg/ml dox in 5% sucrose water) ad libitum. All animal experiments were conducted by following guidelines established by the Institutional Animal Care and Use Committee.

### Histological and Immunohistochemical Analyses

Skin from the dorsal side as well as other tissues was fixed in buffered formalin, embedded in paraffin, sectioned (5 μM), and stained with hematoxylin and eosin. Skin sections were stained with Giemsa stain to detect mast cells, while MPO, CD3, CD163, and Pax-5 antibodies were used for detection of other types of inflammatory cells by immunohistochemical analyses (Carson, et al., Histotechnology: A Self-Instruction Text, 3 ed., American Society for Clinical Pathology Press, Hong Kong, 2009).

RT-PCR and mtDNA Content Analyses

To measure relative gene expression by RT-PCR, total cellular RNA from the skin samples was isolated using Trizol (Invitrogen, Carlsbad, Calif., USA). Approximately, 1000-2000 ng RNA was normalized across samples, and cDNA was generated using the Iscript cDNA synthesis kit (Bio-Rad Laboratories, Hercules, Calif., USA). cDNA was then subjected to RT-PCR using Green Taq PCR mixture (Promega, Madison, Wis., USA) and gene-specific primers as given in Table 1 below. PCR products were run on 1.5 to 2% agarose gel and photographed using gel documentation system. At least three biological replicates were used in each PCR. β2-Microglobulin or RNU6B was used as an internal control in each PCR. mtDNA content analyses in the skin and other tissues were carried out as reported earlier (Singh et al., PloS One, 10, e0139846, 2015).

TABLE 1

| | | |
|---|---|---|
| Target | Forward primer | Reverse primer |

Primers used for genotyping

| | | |
|---|---|---|
| POLG1 | CAA GGT CCA GAG AGA AAC TG | CTC TGT ACC ACC CAA TTC AC |
| | SEQ ID NO: 39 | SEQ ID NO: 40 |
| CAG-rtTA | CTG CTG TCC ATT CCT TAT TC | CGA AAC TCT GGT TGA CAT G |
| | SEQ ID NO: 41 | SEQ ID NO: 42 |
| GFP | GGG CAA TAA GAT GGA GTA CA | TGG ACA GGT AGT GGT TAT CG |
| | SEQ ID NO: 43 | SEQ ID NO: 44 |

Primers used for RT-PCR

| | | |
|---|---|---|
| POLG1 | CCA GGG AGA GTT TAT AAC CA | CAA ATT CCT CAA ACA GCC AC |
| | SEQ ID NO: 3 | SEQ ID NO: 4 |
| COXII | GGC ACC TTC ACC AAA ATC AC | CGG TTG TTG ATT AGG CGT TT |
| | SEQ ID NO: 5 | SEQ ID NO: 6 |
| NDI | CCT ATC ACC CTT GCC ATC AT | TTG CTG CTT CAG TTG ATC GT |
| | SEQ ID NO: 7 | SEQ ID NO: 8 |
| NF-κB | TGG CCG TGG AGT ACG ACA A | GCA TCA CCC TCC AGA AGC A |
| | SEQ ID NO: 9 | SEQ ID NO: 10 |
| MMP2 | ACC TGA ACA CTT TCT ATG GCT | CTT CCG CAT GGT CTC GAT G |
| | G SEQ ID NO: 11 | SEQ ID NO: 12 |
| MMP9 | CTG GAC AGC CAG ACA CTA AAG | CTC GCG GCA AGT CTT CAG AG |
| | SEQ ID NO: 13 | SEQ ID NO: 14 |
| TIMP1 | CTT GGT TCC CTG GCG TAC TC | ACC TGA TCC GTC CAC AAA |
| | SEQ ID NO: 15 | CAG SEQ ID NO: 16 |
| COL1A1 | CTG GCG GTT CAG GTC CAA T | TTC CAG GCA ATC CAC GAG C |
| | SEQ ID NO: 17 | SEQ ID NO: 18 |
| Cyclooxy | AAC CGC ATT GCC TCT GAA T | CAT GTT CCA GGA GGA TGG AG |
| genase 2- | SEQ ID NO: 19 | SEQ ID NO: 20 |
| CCL5 | AGA TCT CTG CAG CTG CCC TCA | GGA GCA CTT GCT GCT GGT GTA |
| | SEQ ID NO: 21 | G SEQ ID NO: 22 |
| IL28a | AGG TCT GGG AGA ACA TGA CTG | CTG TGG CCT GAA GCT GTG TA |
| | SEQ ID NO: 23 | SEQ ID NO: 24 |
| IFNB1 | GTC ATG GGT TTC TCA TGA AGA | CAG ACC CCT TCC AGT GAT TCA |
| | ACA G SEQ ID NO: 25 | TC SEQ ID NO: 26 |
| VEGF | GAG GAT GTC CTC ACT CGG ATG | GTC GTG TTT CTG GAA GTG AGC |
| | SEQ ID NO: 27 | AA SEQ ID NO: 28 |
| IGF1R | CGA GCT TCC TGT GAA AGT GAT | CAC GTT ATG ATG ATT CGG TTC |
| | GT SEQ ID NO: 29 | TTC SEQ ID NO: 30 |
| Klotho | GGA CAT TTC CCT GTG ACT TTG | AGA GAG AGT AGT GTC CAC |
| | C SEQ ID NO: 31 | TTG AAC GT SEQ ID NO: 32 |
| MRPS5 | AAC CAC TGT CTG ACC AGC TTG | AGT CTC TGC TAA TGC GCC TTT |
| | SEQ ID NO: 33 | SEQ ID NO: 34 |
| RNU6B | CTC GCT TCG GCA GCA CA | AAC GCT TCA CGA ATT TGC GT |
| | SEQ ID NO: 35 | SEQ ID NO: 36 |
| B2M | ATG GGA AGC CGA ACA TAC TG | CAG TCT CAG TGG GGG TGA AT |
| | SEQ ID NO: 37 | SEQ ID NO: 38 |

### BN-PAGE and Western Blot Analyses

Mitochondrial isolation was carried out as previously described (Johnstone et al., J Biol Chem, 277, 42197-42204, 2002). To analyze mitochondrial OXPHOS super complexes, Blue-Native polyacrylamide gel electrophoresis (BN-PAGE) was performed with mitochondrial fractions prepared from the skin samples as described previously (Schagger et al., Methods Enzymol, 260, 190-202, 1995). Protein expression of mitochondrial OXPHOS subunits in the skin samples was carried out following standard immunoblots. A premixed cocktail containing primary monoclonal antibodies (Mitosciences, Eugene, Oreg., USA) against subunits of OXPHOS complexes was used to detect OXPHOS super complexes in BN-PAGE analyses and protein expression of OXPHOS subunits in immunoblot analyses. Voltage-dependent anion channel (VDAC) or β-actin antibodies were used as loading controls.

### Analysis of Enzymatic Activities of OXPHOS Complexes

Isolated mitochondria were used for the measurement of enzymatic activities of OXPHOS complexes as previously described (Owens et al., PloS One, 6, e23846, 2011).

### Transmission Electron Microscopy

Transmission electron microscopic analyses of skin samples were carried as described previously (NAG et al., J Mol Cell Cardiol, 15, 301-317, 1983). Images were taken using the FEI-Tecnai electron microscope.

### Cell Culture

Skin fibroblasts from wild-type C57BL/6 (control cells) and mtDNA-depleter mice (POLG1-DN cells) were generated and spontaneously immortalized as described (Todaro et al., J Cell Biol, 17, 299-313 (1963). These cells were maintained in DMEM/F12 (Cellgro, Herndon, Va.) supplemented with 10% FBS (Atlanta Biologicals, Lawrenceville, Ga.). To induce POLG1-DN expression in skin fibroblasts, 1 μg/ml dox dissolved in water was added to the cells in culture and after 6 days of incubation, cells were washed with PBS and collected in Trizol for isolation of total RNA. To estimate cell proliferation and cell survival, MTT assays were carried out as described previously (Ronghe et al., J Steroid Biochem Mol Biol, 144 PtB, 500-512, 2014). Both control and POLG1-DN cells were first treated with dox (1 μg/ml) for 3 days and then cells were plated at a density of 3000 cells/well in 96 well plate with or without dox (1 μg/ml) containing culture media. Readings were taken at every 24 hours.

### STATISTICAL ANALYSES

Statistical analyses were performed using unpaired Student's t test. Data are expressed as mean±s.e.m. P values <0.05 were considered significant. All cellular experiments were repeated at least three times.

1. A transgenic, non-human animal comprising a transgene integrated into a genome of the animal, the transgene comprising an inducible expression control sequence and a first nucleotide sequence encoding a mutant POLG1 polypeptide operably linked to the inducible expression control sequence and optionally a second nucleotide sequence encoding a reporter polypeptide operably linked to the inducible expression control sequence.

2. The transgenic, non-human animal of claim 1, wherein the inducible expression control sequence comprises a tetracycline response element (TRE) and a promoter operably linked to the TRE.

3. The transgenic, non-human animal of claim 1, wherein the transgene comprises the second nucleotide sequence and the inducible expression control sequence comprises a tetracycline response element (TRE) and two promoters operably linked to the TRE.

4. The transgenic, non-human animal of claim 1, wherein the transgene is integrated into the genome at a specific, non-disruptive chromosomal locus or the transgene is integrated into the genome at a random, non-disruptive chromosomal locus.

5. The transgenic, non-human animal of claim 1, wherein the non-human animal is selected from the group consisting of: a non-human primate, a mouse, a rat, a cow, a pig, a goat, and a sheep.

6. The transgenic, non-human animal of claim 1, wherein the mutant POLG1 polypeptide has a sequence of SEQ ID NO: 46 wherein position 1135 of SEQ ID NO: 46 is A, or a sequence that is at least 75% identical to SEQ ID NO: 46 wherein position 1135 of SEQ ID NO: 46 is A or a sequence of SEQ ID NO: 47 wherein position 1164 of SEQ ID NO: 46 is A, or a sequence that is at least 75% identical to SEQ ID NO: 47 wherein position 1164 of SEQ ID NO: 46 is A.

7. The transgenic, non-human animal of claim 1, wherein the mutant POLG1 polypeptide comprises an aspartic acid to alanine substitution at a corresponding aspartic acid residue.

8. The transgenic, non-human animal of claim 1, further comprising a second transgene integrated into the genome of the animal, the second transgene comprising a second expression control sequence and a third nucleotide sequence encoding a transactivator capable of activating the inducible expression control sequence and operably linked to the second expression control sequence, wherein the transactivator is expressed upon activation of the second expression control sequence.

9. The transgenic, non-human animal of claim 8, wherein the second transgene is integrated into the genome at a specific, non-disruptive chromosomal locus or the transgene is integrated into the genome at a random, non-disruptive chromosomal locus.

10. The transgenic, non-human animal of claim 8, wherein the second expression control sequence comprises a transactivator promoter selected from the group consisting of: a constitutive promoter, a tissue-specific promoter, a development-stage-specific promoter, and an inducible promoter.

11. The transgenic, non-human animal of claim 8, wherein the transactivator promoter is a skin-specific promoter selected from the group consisting of: a K5 promoter, a K14 promoter, an involucrin promoter, a tyrosinase promoter, and an α-V integrin promoter.

12. The transgenic, non-human animal of claim 8, wherein the inducer compound is tetracycline or a tetracycline derivative.

13. The transgenic, non-human animal of claim 8, wherein the mutant POLG1 polypeptide comprises an aspartic acid to alanine substitution at a corresponding aspartic acid residue.

14. The transgenic, non-human animal of claim 8, wherein the transactivator is a reverse tetracycline transactivator (rtTA), wherein the rtTA is expressed upon activation of the second expression control sequence and activates the inducible expression control sequence in the presence of an inducer compound, thereby inducing expression of the mutant POLG1 polypeptide, the reporter polypeptide, or both the mutant POLG1 polypeptide and the reporter polypeptide, or a tetracycline transactivator (tTA), wherein the tTA is expressed upon activation of the second expression control sequence and activates the inducible expression control sequence in the absence of an inducer compound, thereby inducing expression of the mutant POLG1 polypeptide, the reporter polypeptide, or both the mutant POLG1 polypeptide and the reporter polypeptide.

15. The transgenic, non-human animal of claim 1, wherein the transgene further comprises a second expression control sequence, an excisable inhibitor sequence, and a third nucleotide sequence encoding a transactivator capable of activating the inducible expression control sequence, wherein the third nucleic acid sequence is operably linked to the second expression control sequence only when the excisable inhibitor sequence is excised.

16. The transgenic, non-human animal of claim 15, wherein the excisable inhibitor sequence is flanked on each side by a recognition sequences for a recombinase.

17. The transgenic, non-human animal of claim 15, wherein the second expression control sequence comprises a transactivator promoter selected from the group consisting of: a constitutive promoter, a tissue-specific promoter, a development-stage-specific promoter, and an inducible promoter.

18. The transgenic, non-human animal of claim 15, wherein the transactivator promoter is a skin-specific promoter selected from the group consisting of: a K5 promoter, a K14 promoter, an involucrin promoter, a tyrosinase promoter, and an α-V integrin promoter.

19. The transgenic, non-human animal of claim 15, wherein the inducer compound is tetracycline or a tetracycline derivative.

20. The transgenic, non-human animal of claim 15, wherein the mutant POLG1 polypeptide comprises an aspartic acid to alanine substitution at a corresponding aspartic acid residue.

21. The transgenic, non-human animal of claim 15, wherein the transactivator is a reverse tetracycline transactivator (rtTA), wherein the rtTA is expressed upon excision of the excisable inhibitor sequence and activation of the second expression control sequence and activates the inducible expression control sequence in the presence of an inducer compound, thereby inducing expression of the mutant POLG1 polypeptide, the reporter polypeptide, or both the mutant POLG1 polypeptide and the reporter polypeptide, or a tetracycline transactivator (tTA), wherein the tTA is expressed upon excision of the excisable inhibitor sequence and activation of the second expression control sequence and activates the inducible expression control sequence in the absence of an inducer compound, thereby inducing expression of the mutant POLG1 polypeptide, the reporter polypeptide, or both the mutant POLG1 polypeptide and the reporter polypeptide.

22. The transgenic, non-human animal of claim 21, wherein after expression of the mutant POLG1 polypeptide, the non-human animal exhibits at least one characteristic selected from the group consisting of: reduced mitochondrial (mt) DNA content, reduced mtDNA copy number, changes in mitochondrial protein expression, reduced expression of mitochondrial oxidative phosphorylation complexes, reduced stability of mitochondrial oxidative phosphorylation complexes, skin wrinkles, hair loss, increased epidermal thickness, epidermal hyperplasia, acanthosis, hyperkeratosis, increased expression of at least one gene selected from the group consisting of: NF-κB, COX-2, INF-β1, CCL5, MMP1, MMP2, MMP9, MMP13, IGF1R, VEGF, and MRPS5, decreased expression of TIMP1 and KLOTHO, increased skin inflammation, and aberrant hair follicles.

23. The transgenic, non-human animal of claim 14, wherein after expression of the mutant POLG1 polypeptide, the non-human animal exhibits at least one characteristic selected from the group consisting of: reduced mitochondrial (mt) DNA content, reduced mtDNA copy number, changes in mitochondrial protein expression, reduced expression of mitochondrial oxidative phosphorylation complexes, reduced stability of mitochondrial oxidative phosphorylation complexes, skin wrinkles, hair loss, increased epidermal thickness, epidermal hyperplasia, acanthosis, hyperkeratosis, increased expression of at least one gene selected from the group consisting of: NF-κB, COX-2, INF-β1, CCL5, MMP1, MMP2, MMP9, MMP13, IGF1R, VEGF, and MRPS5, decreased expression of TIMP1 and KLOTHO, increased skin inflammation, and aberrant hair follicles.

24. A method for screening a therapeutic agent for the treatment of skin wrinkles, the method comprising:
a. providing a non-human animal capable of inducible expression of a mutant POLG1 polypeptide;
b. stimulating the expression of the mutant POLG1 polypeptide, wherein stimulating expression of the mutant POLG1 polypeptide induces skin wrinkles in the non-human animal;
c. administering an agent to the non-human animal either before step b) or after step b);
d. determining the effect of the agent on skin wrinkles in the non-human animal; and
e. comparing the effect of the agent to a control animal, wherein a reduction in skin wrinkles in the non-human animal after administration of the agent indicates the agent is a therapeutic agent for the treatment of skin wrinkles.

25. The method of claim 24, wherein step b) is accomplished by providing an inducer compound to the non-human animal or withholding the inducer compound from the non-human animal.

26. The method of claim 25, wherein the inducer compound is tetracycline or a tetracycline derivative.

27. The method of claim 24, wherein the mutant POLG1 polypeptide comprises an aspartic acid to alanine substitution at a corresponding aspartic acid residue.

28. A method for screening a therapeutic agent for the treatment of hair loss, the method comprising:
a. providing a non-human animal capable of inducible expression of a mutant POLG1 polypeptide;
b. stimulating the expression of the mutant POLG1 polypeptide, wherein stimulating expression of the mutant POLG1 polypeptide induces hair loss in the non-human animal;
c. administering an agent to the non-human animal either before step b) or after step b);
d. determining the effect of the agent on hair loss in the non-human animal; and
e. comparing the effect of the agent to a control animal, wherein a reduction in hair loss in the non-human animal after administration of the agent indicates the agent is a therapeutic agent for the treatment of hair loss.

29. The method of claim 28, wherein step b) is accomplished by providing an inducer compound to the non-human animal or withholding the inducer compound from the non-human animal.

30. The method of claim 28, wherein the inducer compound is tetracycline or a tetracycline derivative.

31. The method of claim 28, wherein the mutant POLG1 polypeptide comprises an aspartic acid to alanine substitution at a corresponding aspartic acid residue.


SEQUENCE LISTING (54 sequences)

SEQ ID NO: 1 - DNA, 33 bp, Artificial Sequence
  synthesized primer
gcatcagcatccatgcggaggttcgctacctgg

SEQ ID NO: 2 - DNA, 33 bp, Artificial Sequence
  synthesized primer
ccaggtagcgaacctccgcatggatgctgatgc

SEQ ID NO: 3 - DNA, 20 bp, Artificial Sequence
  synthesized primer
ccagggagagtttataacca

SEQ ID NO: 4 - DNA, 20 bp, Artificial Sequence
  synthesized primer
caaattcctcaaacagccac

SEQ ID NO: 5 - DNA, 20 bp, Artificial Sequence
  synthesized primer
ggcaccttcaccaaaatcac

SEQ ID NO: 6 - DNA, 20 bp, Artificial Sequence
  synthesized primer
cggttgttgattaggcgttt

SEQ ID NO: 7 - DNA, 20 bp, Artificial Sequence
  synthesized primer
cctatcacccttgccatcat

SEQ ID NO: 8 - DNA, 20 bp, Artificial Sequence
  synthesized primer
ttgctgcttcagttgatcgt

SEQ ID NO: 9 - DNA, 19 bp, Artificial Sequence
  synthesized primer
tggccgtggagtacgacaa

SEQ ID NO: 10 - DNA, 19 bp, Artificial Sequence
  synthesized primer
gcatcaccctccagaagca

SEQ ID NO: 11 - DNA, 22 bp, Artificial Sequence
  synthesized primer
acctgaacactttctatggctg

SEQ ID NO: 12 - DNA, 19 bp, Artificial Sequence
  synthesized primer
cttccgcatggtctcgatg

SEQ ID NO: 13 - DNA, 21 bp, Artificial Sequence
  synthesized primer
ctggacagccagacactaaag

SEQ ID NO: 14 - DNA, 20 bp, Artificial Sequence
  synthesized primer
ctcgcggcaagtcttcagag

SEQ ID NO: 15 - DNA, 20 bp, Artificial Sequence
  synthesized primer
cttggttccctggcgtactc

SEQ ID NO: 16 - DNA, 21 bp, Artificial Sequence
  synthesized primer
acctgatccgtccacaaacag

SEQ ID NO: 17 - DNA, 19 bp, Artificial Sequence
  synthesized primer
ctggcggttcaggtccaat

SEQ ID NO: 18 - DNA, 19 bp, Artificial Sequence
  synthesized primer
ttccaggcaatccacgagc

SEQ ID NO: 19 - DNA, 19 bp, Artificial Sequence
  synthesized primer
aaccgcattgcctctgaat

SEQ ID NO: 20 - DNA, 20 bp, Artificial Sequence
  primer
catgttccaggaggatggag

SEQ ID NO: 21 - DNA, 21 bp, Artificial Sequence
  synthesized primer
agatctctgcagctgccctca

SEQ ID NO: 22 - DNA, 22 bp, Artificial Sequence
  synthesized primer
ggagcacttgctgctggtgtag

SEQ ID NO: 23 - DNA, 21 bp, Artificial Sequence
  synthesized primer
aggtctgggagaacatgactg

SEQ ID NO: 24 - DNA, 20 bp, Artificial Sequence
  synthesized primer
ctgtggcctgaagctgtgta

SEQ ID NO: 25 - DNA, 25 bp, Artificial Sequence
  synthesized primer
gtcatgggtttctcatgaagaacag

SEQ ID NO: 26 - DNA, 23 bp, Artificial Sequence
  synthesized primer
cagaccccttccagtgattcatc

SEQ ID NO: 27 - DNA, 21 bp, Artificial Sequence
  synthesized primer
gaggatgtcctcactcggatg

SEQ ID NO: 28 - DNA, 23 bp, Artificial Sequence
  synthesized primer
gtcgtgtttctggaagtgagcaa

SEQ ID NO: 29 - DNA, 23 bp, Artificial Sequence
  synthesized primer
cgagcttcctgtgaaagtgatgt

SEQ ID NO: 30 - DNA, 24 bp, Artificial Sequence
  synthesized primer
cacgttatgatgattcggttcttc

SEQ ID NO: 31 - DNA, 22 bp, Artificial Sequence
  synthesized primer
ggacatttccctgtgactttgc

SEQ ID NO: 32 - DNA, 26 bp, Artificial Sequence
  synthesized primer
agagagagtagtgtccacttgaacgt

SEQ ID NO: 33 - DNA, 21 bp, Artificial Sequence
  synthesized primer
aaccactgtctgaccagcttg

SEQ ID NO: 34 - DNA, 21 bp, Artificial Sequence
  synthesized primer
agtctctgctaatgcgccttt

SEQ ID NO: 35 - DNA, 17 bp, Artificial Sequence
  synthesized primer
ctcgcttcggcagcaca

SEQ ID NO: 36 - DNA, 20 bp, Artificial Sequence
  synthesized primer
aacgcttcacgaatttgcgt

SEQ ID NO: 37 - DNA, 20 bp, Artificial Sequence
  synthesized primer
atgggaagccgaacatactg

SEQ ID NO: 38 - DNA, 20 bp, Artificial Sequence
  synthesized primer
cagtctcagtgggggtgaat

SEQ ID NO: 39 - DNA, 20 bp, Artificial Sequence
  synthesized primer
caaggtccagagagaaactg

SEQ ID NO: 40 - DNA, 20 bp, Artificial Sequence
  synthesized primer
ctctgtaccacccaattcac

SEQ ID NO: 41 - DNA, 20 bp, Artificial Sequence
  synthesized primer
ctgctgtccattccttattc

SEQ ID NO: 42 - DNA, 19 bp, Artificial Sequence
  synthesized primer
cgaaactctggttgacatg

SEQ ID NO: 43 - DNA, 20 bp, Artificial Sequence
  synthesized primer
gggcaataagatggagtaca

SEQ ID NO: 44 - DNA, 20 bp, Artificial Sequence
  synthesized primer
tggacaggtagtggttatcg

SEQ ID NO: 45 - protein, 1239 aa, Homo sapiens
MSRLLWRKVAGATVGPGPVPAPGRWVSSSVPASDPSDGQRRRQQQQQQQQQQQQQPQQPQVLSSEGGQLRHNPLDIQMLSRGLHEQIFGQGGEMPGEAAVRRSVEHLQKHGLWGQPAVPLPDVELRLPPLYGDNLDQHFRLLAQKQSLPYLEAANLLLQAQLPPKPPAWAWAEGWTRYGPEGEAVPVAIPEERALVFDVEVCLAEGTCPTLAVAISPSAWYSWCSQRLVEERYSWTSQLSPADLIPLEVPTGASSPTQRDWQEQLVVGHNVSFDRAHIREQYLIQGSRMRFLDTMSMHMAISGLSSFQRSLWIAAKQGKHKVQPPTKQGQKSQRKARRGPAISSWDWLDISSVNSLAEVHRLYVGGPPLEKEPRELFVKGTMKDIRENFQDLMQYCAQDVWATHEVFQQQLPLFLERCPHPVTLAGMLEMGVSYLPVNQNWERYLAEAQGTYEELQREMKKSLMDLANDACQLLSGERYKEDPWLWDLEWDLQEFKQKKAKKVKKEPATASKLPIEGAGAPGDPMDQEDLGPCSEEEEFQQDVMARACLQKLKGTTELLPKRPQHLPGHPGWYRKLCPRLDDPAWTPGPSLLSLQMRVTPKLMALTWDGFPLHYSERHGWGYLVPGRRDNLAKLPTGTTLESAGVVCPYRAIESLYRKHCLEQGKQQLMPQEAGLAEEFLLTDNSAIWQTVEELDYLEVEAEAKMENLRAAVPGQPLALTARGGPKDTQPSYHHGNGPYNDVDIPGCWFFKLPHKDGNSCNVGSPFAKDFLPKMEDGTLQAGPGGASGPRALEINKMISFWRNAHKRISSQMVVWLPRSALPRAVIRHPDYDEEGLYGAILPQVVTAGTITRRAVEPTWLTASNARPDRVGSELKAMVQAPPGYTLVGADVDSQELWIAAVLGDAHFAGMHGCTAFGWMTLQGRKSRGTDLHSKTATTVGISREHAKIFNYGRIYGAGQPFAERLLMQFNHRLTQQEAAEKAQQMYAATKGLRWYRLSDEGEWLVRELNLPVDRTEGGWISLQDLRKVQRETARKSQWKKWEVVAERAWKGGTESEMFNKLESIATSDIPRTPVLGCCISRALEPSAVQEEFMTSRVNWVVQSSAVDYLHLMLVAMKWLFEEFAIDGRFCISIHDEVRYLVREEDRYRAALALQITNLLTRCMFAYKLGLNDLPQSVAFFSAVDIDRCLRKEVTMDCKTPSNPTGMERRYGIPQGEALDIYQIIELTKGSLEKRSQPGP

SEQ ID NO: 46 - protein, 1236 aa, Pan troglodytes
MSRLLWRKVAGATVGPGPVPAPGRWVSSSVPASDPSDGQRRRQQQQQQQPQQPQQPQVLSSEGGQPRHNPLDIQMLSRGLHEQIFGQGGEMPGEAAVRRSVEHLQKHGLWGQPAVPLPDVELRLPPLYGDNLDQHFRLLAQKQSLPYLEAANLLLQAQLPPKPPAWAWAEGWTRYGPEGEAVPVAIPEERALVFDVEVCLAEGTCPTLAVAISPSAWYSWCSQRLVEERYSWTSQLSPADLIPLEVPASASSPTQRDWQEQLVVGHNVSFDRAHIREQYLIQGSRMRFLDTMSMHMAISGLSSFQRSLWIAAKQGKHKVQPPTKQGQKSQRKARRGPAISSWDWLDISSVNSLAEVHRLYVGGPPLEKEPRELFVKGTMKDIRENFQDLMQYCAQDVWATHEVFQQQLPLFLERCPHPVTLAGMLEMGVSYLPVNQNWERYLAEAQGTYEELQREMKKSLMDLANDACQLLSGERYKDDPWLWDLEWDLQEFKQKKARKVKKEPATASKLPIEGAGAPGDPMDQEDLGPCSEEEEFQQDVMARACLQKLKGTTELLPKRPQHLPGHPGWYRKLCPRLDDPAWTPGPSLLSLQMRVTPKLMALTWDGFPLHYSERHGWGYLVPGRRDNLAKLPTGTTLESAGVVCPYRAIESLYRKHCLEQGKQQLMPQEAGLAEEFLLTDNSAIWQTVEELDYLEVEAEAKMENLRAAVPGQPLALTARGGPKDTQPSYHHGNGPYNDVDIPGCWFFKLPHKDGNSCNVGSPFAKDFLPKMEDGTLQAGPGGASGPRALEINKMISFWRNAHKRISSQMVVWLPRSALPRAVIRHPDYDEEGLYGAILPQVVTAGTITRRAVEPTWLTASNARPDRVGSELKAMVQAPPGYTLVGADVDSQELWIAAVLGDAHFAGMHGCTAFGWMTLQGRKSRGTDLHSKTATTVGISREHAKIFNYGRIYGAGQPFAERLLMQFNHRLTQQEAAEKAQQMYAATKGLRWYRLSDEGEWLVRELNLPVDRTEGGWISLQDLRKVQRETARKSQWKKWEVVAERAWKGGTESEMFNKLESIATSDIPRTPVLGCCISRALEPSAVQEEFMTSRVNWVVQSSAVDYLHLMLVAMKWLFEEFAIDGRFCISIHDEVRYLVREEDRYRAALALQITNLLTRCMFAYKLGLNDLPQSVAFFSAVDIDRCLRKEVTMDCKTPSNPTGMERRYGIPQGEALDIYQIIELTKGSLEKRSQPGP

SEQ ID NO: 47 - protein, 1218 aa, Mus musculus
MSRLLWKKVAGAKVASGPVPATARWVASSVLDPVPSDGRPPSQMPSSENGQLRLNPLLIQMLSRGLHEQIFGCGGEMPDEAAVQRSVEHLQKHGLWGQPATPLPDVELRLPRLFGGNLDQHFRLLAQKQSLPYLEAAASLLEAQLPPEPKSWAWAEGWTRYGPEGEAEPVAIPEERALVFDVEVCLAEGTCPTLAVAISPSAWYSWCSRRLVEERYSWTSQLSPADLIPLGGSTSASSSTKQDGQEQLVVGHNVSFDRAHIREQYLIQDSRMRFLDTMSMHMAISGLSSFQRSLWMGAKQGKHKNPAAHKARAEVPEESQWSSESSSWDWMDISSANNLADVHNLYVGGPPLEKEPRELFVKGSMRDIRENFQDLMQYCARDVWATFEVFQQQLPLFLERCPHPVTLAGMLEMGVSYLPVNQNWERYLTEAQNTYEELQREMKKSLMDLANDACQLLSGERYKEDPWLWDLEWDLQEFKQKKAKKVKKPASASKLPIEGAGPFGDPMDQEDPGPPSEEEELQRSVTAHNRLQQLRSTTDLLPKRPQHLPGHPGWYRQLCPRLDDPAWAPGPSLLSLQMRVTPKLMALTWDGFPLHYSDSHGWGYLVPGRRDNLTEPPVSPTVESAAVTCPYRAIESLYRKHCLEQGKQQLEPQEVDLAEEFLLTDNSAMWQTVEELGCLDVEAEAKMENSGLSQPLVLPAACAPKSSQPTYHHGNGPYNDVNIPGCWFFKLPHKDGNNYNVGSPFAKDFLPKMEDGTLQAGPGGASGPRALEINKMISFWRNAHKRISSQMVVWLPRSALPRVVTRHPAFDEEGHYGAILPQVVTAGTITRRAVEPTWLTASNARPDRVGSELKAMVQAPPGYVLVGADVDSQELWIAAVLGDAHFAGMHGCTAFGWMTLQGRKSRGTDLHSKTAATVGISREHAKIFNYGRIYGAGQSFAERLLMQFNHRLTRQEAAEKAQQMYAVTKGLRRYRLSADGEWLVKQLNLPVDRTEDGWVSLQDLRMIRREASRKSRWKKWEVAAERAWTGGTESEMFNKLESIAMSDTPRTPVLGCCISRALEPSVVQGEFITSRVNWVVQSSAVDYLHLMLVAMKWLFEEFAIDGRFCISIHDEVRYLVREEDRYRAALALQITNLLTRCMFAYKLGLNDLPQSVAFFSAVDIDQCLRKEVTMDCKTPSNPTGMERRYGIPQGEALDIYQIIELTKGSLEKRSQPGP

SEQ ID NO: 48 - protein, 1216 aa, Rattus norvegicus
MSRLLWKKVAGAKVASGPVPATGRWVSSSVLDPVPSDGQPQSQMPSSENGQLRLNPLHIQMLSRGLHEQIFGCGGDVPDEAAVQRSIEHLRKHGLWGQPTTPLPDVQLRLPRLFGGNLDQHFRLLAQKQSLPYLEAAASLSEAQLPPQPRKWVWAEGWTRYGPEGEAEPVAIPEERALVFDVEVCLAEGTCPTLAVAISPSAWYSWCSRRLVEERYSWTSQLSPADLIPLGVSASASSSTQQDWQEQLVVGHNVSFDRAHIREQYLIQGSRMRFLDTMSMHMAISGLSSFQRSLWMGAKQGKHKTQHPTKRGQKSQKNANGPAISSWDWMDISSANNLADVHNLYVGGPRLAKEPRELFVKGSMRDIRENFQDLMEYCARDVWATFEVFQQQLPLFLERCPHPVTLAGMLEMGVSYLPVNQNWERYLTEAQSTYEELQREMKKSLMELANDACQLLSGERYKEDPWLWDLEWDLQEFKQKKAKKVKKTASASKLPIEGAGPFGDPMDQEDPGPPSEEEELQQNIMAHTRLQQLKSTTDLLPKRPQHLPGHPGWYRKLCPRLDDPAWTPGPSLLSLQMRVTPKLMALTWDGFPLHYSDSHGWGYLVPGRRDNLTELPVSPTEESAAVTCPYRAIESLYRRHCLDQGKQQLETQETDLAEEFLLTDSAMWQTVEELGCLDVEAEATVESSGLSQPLVPPTACAPKTSQPTYHHGNGPYNDVDIPGCWFFKLPHKDGNNYNVGSPFAKDFLPKMEDGTLQAGPGGARGPRALEINKMISFWRNAHKRISSQMVVWLPRSALPRAVTRHPSFDEESHYGAILPQVVTAGTITRRAVEPTWLTASNARPDRVGSELKAMVQAPPGYVLVGADVDSQELWIAAVLGDAHFAGMHGCTAFGWMTLQGRKSRGTDLHSKTAATVGISREHAKVFNYGRIYGAGQSFAERLLMQFNHRLSRQEAADKAQQMYAVTKGLRRYRLSDDGEWLVKQLNVPVDRTEDGWVSLQDLRKIRREASRKSRWKKWEVVTERAWTGGTESEMFNKLESIAMSDTPRTPVLGCCISRALEPSVVQGEFMTSRVNWVVQSSAVDYLHLMLVAMKWLFEEFAIDGRFCISIHDEVRYLVREEDRYRAALALQITNLLTRCMFAYKLGLNDLPQSVAFFSAVDIDQCLRKEVTMDCKTPSNPTGMERKYGIPQGEALDIYQIIELTKGSLEKRSQPGP

SEQ ID NO: 49 - protein, 1200 aa, Xenopus laevis
MNRLLQKGTSLVPSWRTRGCRYRRCSYAPQLHAKPLEMETSQRRMNPLNIQMLSKGFHEQIFRGKQVQHAEEDVQRSITHLKNHELWGQETSTVPDVELQLPKMYGNNIEEHFQILAQKQSLPYLEAANDLLNCQLPAMPQTWAWQSGWTRYTATGEKELVDFPDEKAMVFDVEVCVTEGCCPTLAVAASPQNWYSWCSRRLIEGRYTWSKELLLSDLFPLETSMNCNYMTKNNWTERLVVGHNVSFDRAHIKEQYLIKGSKTRFMDTMSMHMAISGLTGFQRTLWMASKYGKKKGLQEVKQHIKKTRSNFSGSPISSWDWVNISSINNLADVHALYVGGPPLEKEARELFVKGSMSDIRTEFQELMRYCALDVQATHEVFQEQFPLFMERCPHPVTLSGMLEMGVSYLPVNQNWERYLDEAQTSYEELQKEMKKSLMKLANDACQLLTKDAYKEDPWLWDLEWDIQESKQKKTKISKKQKKANEAAESVGNKLVEDHNEDPGPPTEKEESRPSMGKLYLEDLKLKTLPLLPKRNQHLPGHPGWYRKLCPKLEDPDWLPGPGLISLQMRLTPKLMRLTWDGYPLHYSEKHGWGYLVPGRKNNKLNNEEEEEIIPCPYRAIEDIYAEYSKNKTKDGCLSQHSTIPEEFMLTDDNSMWQKVEELSRTEMDLSSEVPATAKAKKRNNSSEHPVKLEMEFDSLPDNHHGNSPCGDVNVSGCWFYKLPHKDGNANNVGSPFAKDFLPKMEDGTLQASTGDSSATRALEINKMISFWRNAHKRISSQMVVWMKKNELHRTITRDPEFDEENKYGAILAQVVSAGTITRRAVEPTWLTASNARADRVGSELKAMVQVPPGYHLIGADVDSQELWIAAILGEAHFAGIHGCTAFGWMTLQGKKSSGTDLHSKTASTVGISREHAKVFNYGRIYGAGQPFAERLLMQFNHRLTQEQAAEKAKQMYAVTKGIRRYILSKEGEWLVEELGISVERGEENSVNLQDLRKIQKDATKRSRRKWNLVSRRIWTGGTESQMFNKLETIAMSPSPKTPVLGCRISRALEPTAVKGEFITSRVNWVVQSSAVDYLHLMLVAMKWLFEAYDIDGRFCISIHDEVRYLVHSKDRYRAALALQITNLLTRCMFASRLGIQDVPQSVAFFSAVDIDKCLRKEVTMDCSTPSNPNGMEKRYGIPQGEALDIYQILKVTKGVL

SEQ ID NO: 50 - protein, 1254 aa, Saccharomyces cerevisiae
MTKLMVRSECMLRMVRRRPLRVQFCARWFSTKKNTAEAPRINPVGIQYLGESLQRQVFGSCGGKDEVEQSDKLMELSKKSLKDHGLWGKKTLITDPISFPLPPLQGRSLDEHFQKIGRFNSEPYKSFCEDKFTEMVARPAEWLRKPGWVKYVPGMAPVEVAYPDEELVVFDVETLYNVSDYPTLATALSSTAWYLWCSPFICGGDDPAALIPLNTLNKEQVIIGHNVAYDRARVLEEYNFRDSKAFFLDTQSLHIASFGLCSRQRPMFMKNNKKKEAEVESEVHPEISIEDYDDPWLNVSALNSLKDVAKFHCKIDLDKTDRDFFASTDKSTIIENFQKLVNYCATDVTATSQVFDEIFPVFLKKCPHPVSFAGLKSLSKCILPTKLNDWNDYLNSSESLYQQSKVQIESKIVQIIKDIVLLKDKPDFYLKDPWLSQLDWTTKPLRLTKKGVPAKCQKLPGFPEWYRQLFPSKDTVEPKITIKSRIIPILFKLSWENSPVIWSKESGWCFNVPHEQVETYKAKNYVLADSVSQEEEEIRTHNLGLQCTGVLFKVPHPNGPTFNCTNLLTKSYNHFFEKGVLKSESELAHQALQINSSGSYWMSARERIQSQFVVPSCKFPNEFQSLSAKSSLNNEKTNDLAIIIPKIVPMGTITRRAVENAWLTASNAKANRIGSELKTQVKAPPGYCFVGADVDSEELWIASLVGDSIFNVHGGTAIGWMCLEGTKNEGTDLHTKTAQILGCSRNEAKIFNYGRIYGAGAKFASQLLKRFNPSLTDEETKKIANKLYENTKGKTKRSKLFKKFWYGGSESILFNKLESIAEQETPKTPVLGCGITYSLMKKNLRANSFLPSRINWAIQSSGVDYLHLLCCSMEYIIKKYNLEARLCISIHDEIRFLVSEKDKYRAAMALQISNIWTRAMFCQQMGINELPQNCAFFSQVDIDSVIRKEVNMDCITPSNKTAIPHGEALDINQLLDKSNSKLGKPNLDIDSKVSQYAYNYREPVFEEYNKSYTPEFLKYFLAMQVQSDKRDVNRLEDEYLRECTSKEYARDGNTAEYSLLDYIKDVEKGKRTKVRIMGSNFLDGTKNAKADQRIRLPVNMPDYPTLHKIANDSAIPEKQLLENRRKKENRIDDENKKKLTRKKNTTPMERKYKRVYGGRKAFEAFYECANKPLDYTLETEKQFFNIPIDGVIDDVLNDKSNYKKKPSQARTASSSPIRKTAKAVHSKKLPARKSSTTNRNLVELERDITISREY

SEQ ID NO: 51 - protein, 1018 aa, Schizosaccharomyces pombe
MFYKACPSTLTCSKWIHSIIKTKKFLYCRHYSSKSFIDNAPLRINPVGVQYLSPALQNQVFPQQNTQISQLHLDLAKFHLAKHQLLNKETIKLPSFNFRLPPLQGKTISEHFYNIGLEFAEPHLSKAIKFSKIDTPVQPKTWKRQPGWTKYAKDGSISCVPYPDSDCMVFDVEVLYKVSPFAVVATAVSEDAWYCWLSPWLLGKSENDRQLIPSNPKGALFVGHNVSFDRQRIREEYNIKSSRNVFLDTMSLHVATHGMCSRQKPTWFKARKAYIRSQSTETSEDDDSSSFDDDYQNYLKQEPWLAHSSVNSLKDVAKFHCNITLDKSKRDDFASLEKEPILQKLNELITYCAHDTYSTHQVFKKVFPQFLEVCPHPATFSAMLSLGSVFLPVNHSWTRYINGVEEQYQQMIQLVDQKLSQYAEKAKDLINTKDTVLKDPWLRQLDWTPCNLYRKLKKATQEVPVVPKWYKKAYCKTEKRAVITAKSRLAPILLRLKWKKHPLAWSDTYGWVFSVERTSKDEIEMLLDQGLVPCSREEDTKLDYNNYIFFKVPHKDGPEARCGSPLSKSYQRYFEEGILQSDYEVAKKALEMSASCSYWSSARDRIRSQMVVWDKDAELGVPSSVDGFGIILPCIIPMGTVTRRAVENTWLTASNSKKNRLGSELKAMIRAPDGYTFVGADVDSEELWIVALMGDSQFRLHGATALGMMTLEGKKSEGTDLHSKTAAILGVSRDSAKVFNYGRLYGAGLKHTTLLLMQMNPTLKTAEAKELAKKLYASTKGVKSKMSKRLQEMGLPKLTFWSQGTESFVFNKLEAMAQLPSPRTPVLDAGITQALSSKNLSKNSFMTSRVNWAIQSSAVDYLHLLLVSMNHLIKKYYLEARLSLTVHDEVRYLSSDKDKYRVAFALQVANLWTRAFFCQRLGINELPQSVAFFSSVDIDHVLRKDVKMDCVTPSNKVPIPPGEELTIESVLEKLEQSGQSLEPLEQIQCFVDVKATTSAEITEEDKKNIAYLKAQAFY

SEQ ID NO: 52 - protein, 1456 aa, Neurospora crassa
MLTPVRCRTVPNATVATAARVLRRANLFSRYPRQLGHLRWDSTIAQVLERKGLGVPSTARHNEIGVQQLSEHLYKQLFPRGNTDPPAPELIELAKDHLARHDLLGKTTDKTPPIAFQLPALVGDTLDEHFHKLGVDAAEPFLTHAKQFADAHLPPKPTSWVRRSGWTKYNRDGTTENDVLPQGNMMCFDVEVMYKDNPYAVMACAGTPDAWYAWLSPWLLGETENKAQLVPMGDPTVDRIIVGHNIGYDRAKILEEYDLKQTRNFFLDTMSLHVAVNGMCSQQRPTWMKHKKARELREKAEHESASVELQEVLQGGSLTAEEADLWVDKSSINSLRDVAQFHLNVKIDKDIRDVFAETDRNVILNQLDDLLTYCAADVQVTHQVYQVVFPNFLGVCPHPVSFAALRHLASVILPVNKTWDTYIETAEATYLQMLHGVQERLFTLMERTLDYKADPEKYLSDPWLSQLDWSGQEIKMAKPKKKGDVERPALNQKLPGYPQWYKDLFVKVPKELSGLDEPDKEQENRKARHEFINLTVRSRIAPLLLKLSWEGYPLFWSDQFGWTFQVPREKAETFIQRQMTPVQFEDPDVDDRLRMDVDHKYFKLPHKDGPNARCVNPMAKGYLPYFEKGILSSEYPYAKEALEMNASCSYWISARERIKNQMVVYEDQLPPSQRFVNKDADSNTPIGGFVLPQVIPMGTITRRAVERTWLTASNAKKNRVGSELKAMVRAPPGYVFVGADVDSEELWIASVVGDATFKLHGGNAIGFMTLEGTKSQGTDLHSRTASILGITRNDAKVFNYGRIYGAGLKFASQLLRQFNPSLTEAETTAIATKLYDATKGAKTNRKSLYKRPFWRGGTESFVFNMLEEFAEQERPRTPVLGAGITEALMSRWVSKGGFLTSRINWAIQSSGVDYLHLLIIAMDYLTRRFNLACRLAITVHDEIRYLAEEPDKYRVAMALQIANLWTRVMFAQQVGIQDLPQSCAFFSAVDIDHVLRKEVDMDCITPSNPIPIAHGESIDIFQILEKGDDAKLDDSIVPQSQYAPRLENIPYTPRVPVMQRLRERAEAGDHQAFLRFIRAQITNSDEELKRIIAETRYSDPYGAFSLASNGRVSGNPHQRHAAVHASTKTAAAPSKPSIASRFDSVSQASRIKSVAAGSDEPTIRATKAQGKAMAKASGTKLAASTKDTVLNVTIKKKVAAPEMAAVPSTSSESKSKASATTSTTTTENATASPSSSSNVDAKKTTSKTKPTHKKETEGEPFPSLDDPVIAARLEAVSKTSPGTRASVAAKLDALASFSMRAAAAAEAAVTTTTTPEPPTNPPPVAPKAKEPTTTMAEKVLKPTAVPKNPTPTLTPTTKKSNPTSTPTTPKPVGRPRTTPILPYTAPKKKLPSSAFSKEQKEPMSVSEAVWTASVGGRATTTTATATATGDEKKKTTTTPSWKPVKESLFGRGVYIP

SEQ ID NO: 53 - protein, 1197 aa, Maylandia zebra
MLHVLRCPLKRTRISLQWMSLRGFCSTKPHILQGEDSTQTRLNPLNIQMLSKGLHEQIFRGLEPEYREEAVQRSIRHLQKHQLWGKDTSLLPDVDLKLPKMYGNDIDEHFCHLAQTQSLPYLEAATKLKLADLPPMPQEWSWEVGWTRYGPTGESQKVDFPDESALVFDVEVCTTEGQCPTLAVAVSPTNWYSWCSKRLIEERYSWSNHLTLADLIPLETPFNSARPPGGKWKERLIVGHNVSFDRAHIKEQYLLKGSKVRFMDTMSLHMAISGLTGFQRTLWMASKLGKKRGLQEVKEHIKKAGKKHEGPMIGSWDWVNISSINNLADVHALYVGGPPLQKEARETFVKGSMTDVRNNFQELMQYCALDVKATHEVFTEQLPLFMERCPHPVTLAGMLEMGVSYLPVNQNWGRYLEDSQDVYEELQREMKKSLMILADDACQLLENDRYKEDPWLWDLDWDVQEFKQKKVAASKKKGSKKADNKQIATPLPDWEEDPGPPSEEEMEGPRPSRLAVENLKETVNRLPKRRQHLPAHPGWYRKLCEKMSDDSWSPGASLISLQMRLTPKLMGLTWDGFPLHYTEKHGWGYLVPGRRDNLDSQEENAGPVCPHRAIESVYKEYCEQHSKERPDYLDSIPSDDLMLTDSTVWTKVEELSSLESQMEENALRMRKSAEKNKNRSTDPHYMKEGSHCHYHHGNGPYNDVDIPGCWFFKLPHKDGNHNNVGSPFSKDFLSKMEDGTLRAGRGGTNATRALEINKMMSFWRNAHKRISSQMVLWLRKVELPRSVSRHKDFDEEGQYGAILPQVVTAGTVTRRAVEPTWLTASNARRDRVGSELKAMVQVPPGYHLIGADVDSQELWIAAVLGEAHFAGMHGCTAFGWMTLQGKKSEGTDLHSRTADTVGISREHAKVFNYGRIYGAGQPFAERLLMQFNHRLSQTEAASKARQMYALTKGIRRYHLSEDGEWLVNELGIDVEREEDGSVSLQELRRITRVASQSSRRKRWDIVGKRLWAGGTESDMFNKLESIAHSKNPATPVLGCRISRALEPNAVKDEFITSRVNWVVQSSAVDYLHLMLVVMKWLIEEYNIDGRFCISIHDEVRYLVRSEDCYRAALALQITNLLTRSMFAHALGMQDLPQSVAFFSAVDIDQCLRKEVNMDCVTPSNPTGVERKYGLPPGEALDIYQIIDITKGSLNKGR

SEQ ID NO: 54 - protein, 1206 aa, Danio rerio
MLRLISCHRWQTFVRSGCWVRRCSTSVKSKPQQGSDQTRLNPLNIQMLSRNLQEQIFRGQTQEYTDEDVERSIRHLEHHGLWGKETSLLPDVQLRLPEMYGSNIDEHFRLLAQKQSLPYLEAASQLQKAQIPEMPQEWAWEVGWTRYGADGESQKVNFPDENALVFDVEVCMAEGHCPTLAVALSPTAWYSWCSHRLVEDRYAWSSDLSLADLIPLETLANSSQPRGGEWKERLVVGHNVSFDRAHIKEQYLLKGSKMRFLDTMSLHMAISGLTGFQRSLWMASKYGKRKGLQEVKEHMKRLGRRPEGPKIGSWEWVNISSINNLADVHALYVGGDPLMKESRELFVKGSMSDIRGNFQELMQYCALDVLATQEVFTQQLPLFMERCPHPVTLAGMLEMGVCYLPVNHNWRRYLEDAQATYEELQREMKKSLMILADDACQLLQDDRYKDDPWLWDLDWDVQEFKQKKVPVSKKKAKQEAEATEAANDSEAIDWPEDPGPPLEEEEGDSDPRRELLQRLKETVSCLPKRRQHLPAHPSWYRKLCVKMSEAEDWSPGASLISLQMRITPKLMGLTWDGFPLHYTDQHGWGYLVPGRRDNLDISEDNEEPVCPYRAIESVYKEYCEQKGKEQPKYPDSPVSDELMLTDTAVWQTVEEVSRLEMFDDEALSTAAPSKRITKKVNSVFKSLNGECPYHHGNGPYNDVNIPGCWFFKLPHKDGNENNVGSPFSKDFLSKMESGTLQAGREGTNATRALEINKMISFWRNAQKRISSQMVVWLRRAELPRSVRLHQDYDEEGQYGAILPQVIPAGTVTRRAVEPTWLTASNARKDRVGSELKAMVQVPPAYHLVGADVDSQELWIAAMLGEAHFAGMHGCTAFGWMTLQGKKSQGTDLHSRTADAVGISREHAKVFNYGRIYGAGQPFAERLLMQFNHRLSQQEAAGKARQMYALTKGLRRYHLSEEGEWLLQKLNVNVERAEDGSISLQDLRKISKVATQSTTRGSKKKWNLTAGRVWTDGTESYMFNKLESIAQSDRPATPVLNCRISRALEPSAVQEEFITSRVNWVVQSSAVDYLHLMLVSMRWLFEEHDIDGRFCISIHDEVRYLVTSEDRYRAALALQITNLLTRCMFAFKLGMMDLPQSVAFFSAVDIDKCLRKEVTMDCKTPSSPAGVERRYGLPQGEALDIYQIIEITKGSLTKEKRQAPRSK